(19) United States
(12) Patent Application Publication
(10) Pub. No.: US 2023/0295575 A1
(43) Pub. Date: Sep. 21, 2023

(54) METHODS OF INCREASING MATURATION OF HEART, PANCREATIC BETA-CELLS, AND NEURONS

(71) Applicant: Yissum Research Development Company of the Hebrew University of Jerusalem Ltd., Jerusalem (IL)

(72) Inventors: Yaakov Nahmias, Mevaseret Zion (IL); Avner Ehrlich, Jerusalem (IL); Muneef Ayyash, Jerusalem (IL)

(21) Appl. No.: 17/760,378

(22) PCT Filed: Feb. 9, 2021
(86) PCT No.: PCT/IB2021/000106

§ 371 Date: Aug. 9, 2022

Related U.S. Application Data
(60) Provisional application No. 62/972,001, filed on Feb. 9, 2020.

(51) Int. Cl. C12N 5/077; G01N 33/50

(52) U.S. Cl. CPC C12N 5/0657; G01N 33/5014; C12N 2500/36; C12N 2500/98; C12N 2506/45; G01N 2333/916

(57) ABSTRACT

Provided are methods of generating a metabolically mature human cell selected from the group consisting of a cardiomyocyte, a pancreatic beta cell, and a neuronal cell, using an effective concentration of a conjugated fatty acid and optionally a nonconjugated fatty acid selected from the group consisting of: a monounsaturated omega-9 fatty acid, palmitic acid, linoleic acid (LA) and a short chain fatty acid. Also provided are isolated populations of metabolically mature human cells, and methods and kits using same for selecting a compound for toxicity to the cells.

### CROSS REFERENCE TO RELATED APPLICATION

This application is a §371 of International Application No. PCT/IB2021/000106, filed Feb. 9, 2021, which claims priority to U.S. Provisional Pat. No. 62/972,001 filed Feb. 9, 2020, the entire contents of each being incorporated herein by reference as though set forth in full.

### SEQUENCE LISTING STATEMENT

The ASCII file, entitled SEQLIST.txt, created on Feb. 9, 2021, comprising 9412 bytes, submitted concurrently with the filing of this application is incorporated herein by reference.

### FIELD AND BACKGROUND OF THE INVENTION

The present invention, in some embodiments thereof, relates to methods of increasing the metabolic maturation of a human cell selected from the group consisting of a cardiomyocyte, a pancreatic beta cell and a neuron, and, more particularly, but not exclusively, to methods and kits using metabolically mature human cells.

Mammalian organs continue their development during the weeks and months after birth. Postpartum development is thought to be driven by established transcriptional programs ¹⁻⁴, accommodating the changing demands of the neonate ^{5,6}. One of the most significant changes occurring after birth is a rapid transition from in utero placental nutrition, where oxygen and glucose are delivered directly to the fetal circulation ⁷, to post-partum metabolism where blood flow increases due to changing cardiac capacity, delivering oxygen and lipid-rich nutrition from the rapidly colonizing gut to the neonatal liver ⁸.

Recently, the present inventors showed that microbiome-modified bile acids produced during the postnatal stage, activate PXR-dependent drug metabolism in fetal human hepatocytes ¹². Other work demonstrated that the microbiome plays a role in the development of the muscles, brain and the immune system in mice ¹³⁻¹⁵. These results suggest that the interplay between nutrients and the microbiome may play an important role in the maturation of mammalian organs. Fatty acids may also play a role in neonatal development as their availability changes dramatically during breastfeeding. Neonate supply of long chain polyunsaturated fatty acids (PUFA) increase considerably after birth ¹⁶, driving the maturation of the retina and brain in rodents ¹⁷⁻¹⁹. Regretfully, animal models provide limited insights into human development as there are major differences in the composition of breast milk between mammalian species. There is a significant difference between rodent and human metabolism due to difference in fatty acids types in breast milk, to genetic and metabolic regulatory differences in lipid metabolism.

Human embryonic stem cells are an important model to study human development ^{20,21}. Previous work demonstrated that successful differentiation of liver and cardiac cells must mimic distinct developmental stages seen in vivo ^{22,23}. While current protocols produce homogenous populations of cells, the phenotype of stem cell-derived hepatocytes and cardiomyocytes is still fetal ^{24,25}. Indeed, stem cell-derived hepatocytes and cardiomyocytes remain primarily glycolytic and express fetal markers in spite of significant efforts to enhance maturation.

Additional background art includes Xiaojie Ma and Saiyong Zhu, Acta Biochim Biophys Sin, 2017, 49(4), 289-301; Ulf Diekmann et al., Nature, Scientific Reports (2019) 9:996; Xiulan Yang et al., Stem Cell Reports Vol. 13, 657-668, Oct. 8, 2019; Fabio Bianchi et al., Stem Cell Research 32 (2018) 126-134; Yuya Kunisada et al., Stem Cell Research (2012) 8, 274-284; and U.S. Patent Application Publication No. 2017-0266145-A1.

### SUMMARY OF THE INVENTION

According to an aspect of some embodiments of the present invention there is provided an in vitro method of generating a metabolically mature human cell. An exemplary method includes culture of said human stem cell in xeno-free media in a differentiation medium for an effective amount of time to induce differentiation of the stem cell into an immature differentiated cell, in the absence of a trans fatty acid; followed by incubating the immature cell so differentiated in a maturation medium comprising an effective amount of a conjugated trans fatty acid for a suitable time period, to induce metabolic maturation, thereby producing metabolically mature human cells containing trans fatty acids. In certain embodiments, the maturation medium of step b) further comprises a fatty acid selected from the group consisting of a monounsaturated omega-9 fatty acid, palmitic acid, linoleic acid (LA), or a short chain fatty acid. In preferred embodiments, the trans fatty acid is cis-9, trans-11 conjugated linoleic acid (9CLA). In particularly preferred embodiments, the stem cells are matured into a cell selected from a beating cardiomyocyte, an insulin secreting pancreatic beta cell or a neuronal cell capable of neurotransmission. In certain embodiments, the maturation medium increases spare mitochondrial capacity by at least 60% in the immature differentiated cell so cultured, as measured by seahorse assay. In other embodiments, a metabolically mature cell is defined by a spare mitochondrial capacity which is equal to, or greater than its basal respiration, as measured by seahorse assay. In certain embodiments, a demethylation promoting agent is used in step a) to induce differentiation of said stem cells. Cells can be incubated in differentiation media in step a) at least 30 days. In certain aspects stem cells are cultured in step a) for 8-10 days, 7-30 days, 12-21 days, and 12-30 days. In step b), cells are incubated in maturation medium for 3, 4, 5, 6, 7, 8, 9, or 10 days. In certain embodiments, cells are incubated in maturation medium for 4 days. In other embodiments, the entire maturation process of steps a) and step b) is completed within 21 days.

Mitochondrial spare capacity can be determined using a commercially available seahorse assay.

In certain aspects of the invention, the metabolically mature differentiated cell is a human cardiomyocyte exhibiting a mitochondrial network distributed in the cytosol of said cell rather than being confined to the perinuclear space, as observed in metabolically immature cardiomyocyte. In other aspects, the maturation medium comprises basal media supplemented with B27 supplement minus insulin (1X), oleic acid, and 9CLA. Metabolically mature differentiated human cardiomyocyte can be characterized by at least one of i) sarcomeres of 2.0 to 2.4 µm in length; and ii) a reduced expression by at least 5 fold of a fetal marker selected from the group consisting of: Atrial natriuretic peptide (ANP), Brain Natriuretic Peptide (BNP), Hyperpolarization Activated Cyclic Nucleotide Gated Potassium Channel 1 (HCN1), MYH7 (myosin heavy chain 7), MYH6, cardiac titin (N2B), cardiac troponin I (TNNI3), and sarcoplasmic reticulum ATPase (SERCA2) as compared to the expression of said fetal marker in a human metabolically immature cardiomyocyte obtained in step (a) as measured by an QPCR or RNASEQ analysis.

In other embodiments, the metabolically mature differentiated cell is a human pancreatic beta cell is characterized by at least a two-fold increase in insulin secretion in response to glucose stimulation as compared to insulin secretion in response to glucose stimulation in an immature differentiated human pancreatic beta cell obtained in step (a) under identical conditions. In certain aspects, the maturation medium for generating pancreatic beta cells comprises of basal media supplemented with Alk5i II, T3, oleic acid, and 9CLA.

In another embodiment, the metabolically mature differentiated cell is a human neuronal cell, characterized by a mitochondrial spare capacity and a basal respiration rate of at least 40% above a spare capacity and a basal respiration rate observed in neuronal fetal cells isolated from a human fetal brain of a gestation week of 16-24 weeks, or stressed neuronal cells, as measured by seahorse assay, and a reduced expression by at least 5-fold of a fetal marker selected from the group consisting of: Cyclin B2 (CCNB2), Glial fibrillary acidic protein (GFAP), Oligodendrocyte Transcription Factor 1 (OLIG1) and Stathmin 2 (STMN2) as measured by an QPCR or RNASEQ analysis as compared to the expression of said fetal marker in said neuronal fetal cell or stressed neuronal cell.

According to an aspect of some embodiments of the present invention there is provided an isolated population of metabolically mature human cardiomyocyte cells wherein said population is homogeneous or at least 50% of the cells comprise the metabolically mature human cardiomyocyte cell of some embodiments of the invention.

According to an aspect of some embodiments of the present invention there is provided an isolated population of metabolically mature human pancreatic beta cells wherein said population is homogeneous or at least 50% of the cells comprise the metabolically mature human pancreatic beta cell of some embodiments of the invention.

According to an aspect of some embodiments of the present invention there is provided an isolated population of metabolically mature human neuronal cells wherein said population is homogeneous or at least 50% of the cells comprise the metabolically mature human neuronal cell of some embodiments of the invention.

According to an aspect of some embodiments of the present invention there is provided a method of selecting a compound which is toxic to cells, comprising:
(a) incubating the isolated population of cells of claim 34, 35 or 36 with the compound for a pre-determined time period, and;
(b) measuring following said pre-determined time period a level of metabolic activity selected from the group consisting of: an intracellular esterase activity and a conversion of MTT 3-(4,5-dimethylthiazol-2-yl)-2,5-diphenyltetrazolium bromide to insoluble formazan, wherein a decrease in said level below a predetermined threshold indicates that said compound is toxic to the cells,
thereby selecting a compound which is toxic to cells.

According to an aspect of some embodiments of the present invention there is provided a method of selecting a compound which is toxic to cells, comprising:
(a) incubating a predetermined number of cells from the isolated population of cells of some embodiments of the invention with the compound for a pre-determined time period, and;
(b) counting the number of cells following said pre-determined time period, wherein a decrease in said number of cells below a predetermined threshold following said incubating with the compound indicates that said compound is toxic to the cells,

According to an aspect of some embodiments of the present invention there is provided a kit for screening a compound which is toxic to cells, the kit comprising the isolated population of cells of some embodiments of the invention and at least one agent capable of detecting a toxicological end-point selected from the group consisting of: a cell viability assay, a functional viability assay, a calcium handling assay, an inflammation/injury marker assay or any other standard assay published by TOX21, EuroTOX, EPA or any other governmental agency.

According to some embodiments of the invention, the culture conditions further increase a basal respiration rate of said metabolically immature cell by at least 60% above a basal respiration rate characterizing said metabolically immature cardiomyocyte, said metabolically immature pancreatic beta cell, or said metabolically immature neuron resultant of step (a) as measured by seahorse assay.

According to some embodiments of the invention, the human pluripotent stem cell is a “methylated human pluripotent stem cell” characterized by a genomic DNA having at least 60% methylated CpG dinucleotides in CpG islands present in the genomic DNA, wherein said CpG island is composed of at least 200 nucleotides of which more than 50% are CpG dinucleotides.

According to some embodiments of the invention, the method further comprises a step of contacting said human pluripotent stem cell with an effective concentration of a demethylation promoting agent prior to subjecting said human pluripotent stem cell to conditions suitable for differentiating said human pluripotent stem cell into said metabolically immature cell, wherein said contacting is in the absence of said conditions suitable for said differentiating.

According to some embodiments of the invention, the contacting occurs for at least 20-24 hours prior to subjecting said human pluripotent stem cell to said conditions.

According to some embodiments of the invention, the human pluripotent stem cell is a human induced pluripotent stem cell derived from a somatic cell of an adult human subject being at least 8 year-old.

According to some embodiments of the invention, the human pluripotent stem cell is a human embryonic stem cell obtained following at least 50 passages.

According to some embodiments of the invention, the monounsaturated omega-9 fatty acid is oleic acid (OA).

According to some embodiments of the invention, the conjugated fatty acid is metabolized by the Bifidobacterium and/or lactobacillus bacterial strain(s).

According to some embodiments of the invention, the culturing in claim 1 step (a) is performed in the presence of a culture medium which comprises no more than 0.007 picomolar insulin.

According to some embodiments of the invention, the culturing in claim 1 step (a) is performed in the presence of a culture medium which comprises no more than 0.05 picomolar of cortisone.

According to some embodiments of the invention, metabolically mature cells including cardiomyocytes, neurons or pancreatic beta-cells contain an effective amount of conjugated fatty acids (e.g. 9CLA) in cellular membranes and lipid droplets. According to some embodiments of the invention, metabolically mature cells are characterized by interconnected mitochondrial network covering over 50% of the cellular cytoplasm. According to some embodiments of the invention, metabolically mature cells are characterized by spare mitochondrial capacity that is equal or greater than the cell basal respiration. According to some embodiments of the invention, metabolically mature cells are characterized by 30% decrease extracellular acidification rates over immature cells.

According to some embodiments of the invention, the metabolically mature human cardiomyocyte is characterized by a reduced expression by at least 5 fold of a fetal marker selected from the group consisting of: Atrial natriuretic peptide (ANP), Brain Natriuretic Peptide (BNP), Hyperpolarization Activated Cyclic Nucleotide Gated Potassium Channel 1 (HCN1), myosin heavy chain 6 (MYH6), cardiac titin (N2B), skeletal troponin I (TNNI1), Cyclin-dependent kinase 1 (CDK1), Aurora Kinase B (AURKB) as compared to the expression of said fetal marker in a human metabolically immature cardiomyocyte obtained in step (a) as measured by an QPCR or RNASEQ analysis.

According to some embodiments of the invention, the mature human cardiomyocyte are characterized by anisotropic cells with sarcomere length above 1.9 µm, while immature cardiomyocytes show sarcomere length of 1.5 to 1.8 µm.

According to some embodiments of the invention, the metabolically mature human pancreatic beta cell is characterized by an increased insulin secretion in response to glucose stimulation by at least 2 fold as compared to insulin secretion in response to glucose stimulation in a metabolically immature human pancreatic beta cell obtained in step (a) under identical conditions.

According to some embodiments of the invention, the effective concentration of said conjugated fatty acid in said culture medium is between 10-50 micromolar.

According to some embodiments of the invention, the conjugated fatty acid is cis-9, trans-11 conjugated linoleic acid (9CLA).

According to some embodiments of the invention, the effective concentration of said monounsaturated omega-9 fatty acid is between 50-150 micromolar.

According to some embodiments of the invention, the effective concentration of said oleic acid (OA) is between 50-150 micromolar.

According to some embodiments of the invention, the effective concentration of said oleic acid (OA) is about 100 micromolar.

According to some embodiments of the invention, the effective concentration of said Palmitic Acid is between 50-150 micromolar.

According to some embodiments of the invention, the effective concentration of said linoleic acid (LA) is between 50-150 micromolar.

According to some embodiments of the invention, the effective concentration of said short chain fatty acid is between 500-10,000 micromolar.

According to some embodiments of the invention, the culture medium is devoid of serum.

According to some embodiments of the invention, the culture medium is a chemically defined medium.

According to some embodiments of the invention, the culture medium is devoid of supplemented Carnitine.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-F: Breast milk abundant fatty acids drive cardiac postnatal maturation. FIG. 1A - GeneChip analysis of mRNA isolated from cardiac tissue of 6- to 10-week-old germ free mice (GF), mice that have been conventionalized (CONV-D) and Ppara^{-/-} knockout animals (Ppara^{-/-}). GF and Ppara^{-/-} mice showed significantly lower levels of mature structural, functional and metabolic genes, as well as elevated markers of fetal heart tissue. FIG. 1B - Gene ontology analysis of GeneChip comparing GF and CONV-D mice hearts shows disruption in metabolic pathways, particularly lipid metabolism, glycolysis, and oxidation processes in the mitochondria (p<1.1×10⁻⁴). PPAR signaling pathway in disrupted by the absence of a microbiome in the weeks after birth (p<2.2×10⁻⁵). FIG. 1C - Schematic depicting 11-day differentiation of induced pluripotent stem cells to early-stage neonatal cardiomyocytes, followed by 4-day exposure to breast milk abundant fatty acids and their microbial derivates. FIG. 1D -Seahorse MitoStress analysis of stem cell-derived cardiomyocytes. Fatty acid stimulation mimicking postnatal nutrition increased the oxidative phosphorylation of stem-cell derived cardiomyocytes, as well as maximal respiratory capacity by 1.8- to 2.5-fold (n = 3, p<0.001). Glycolysis decrease by 24% indicating of a more mature metabolic phenotype (Piquereau, J. et al. Maturation of Cardiac Energy Metabolism During Perinatal Development. Front Physiol 9, 959, 2018). Short-term exposure to GW9662 reversed both effects. Upper graph: OCR (Oxygen Consumption Rate) measured in pmol per minute per 10⁴ cells; Lower histogram: quantification of Oxidative (Basal) Respiration, Maximal Respiration Capacity, Spare Respiration Capacity and Glycolysis rate calculated from OCR and ECAR measurements during MitoStress Test. Each Phase is displayed on the top panel. FIG. 1E - qRT-PCR analysis showing up-regulation of key genes related to structural, functional and metabolic maturation of human cardiac tissue, as well as marked down-regulation of fetal genes (n = 3; p<0.01). FIG. 1F - Analysis of cell area of stem cell-derived cardiomyocytes cultured in maturation medium (Control), supplemented with OA+9CLA in the absence or presence of GW9662, PPARα/γ inhibitor (OA+9CLA+GW9662). 9CLA matured cardiomyocytes are hypertrophic, showing a 1.5-fold increase in cell area, reversed by PPARα/γ inhibition. *p<0.05, **p<0.01, ***p<0.001. Error bars represent ± S.E. Significance was determined using a one-tailed heteroscedastic student-t-test.

FIGS. 2A-C - Germ-free mice show impaired cardiac maturation. FIG. 2A - Gene ontology enrichment network map of significantly differentially expressed patterns and KEGG pathways between fetal mouse heart and mature (2-3 months old) mature heart. There is a significant upregulation in transcriptional and signaling processes, metabolism, structural and calcium handling and conduction pathways and a significant downregulation of cell cycle pathways. FIG. 2B - Pathway annotation of significant differentially down regulated genes in germ free mice hearts. Many mature cardiac function related pathway families are differentially expressed in GF hearts, suggesting significant pathways are induced by the emergence of the microbiome postnatally. FIG. 2C - Volcano plot summarizing the differential regulation in germ free and PPARα knockout mice (both compared to conventional wild type mice; CONV WT) hearts. Red, green, and black dots represent genes with higher, lower, or unchanged expression, respectively. Both deficiencies display similar differential expression distribution patterns, suggesting PPAR, and PPARα in particular, play a significant role in maturation caused by the emergence of the microbiome.

FIGS. 3A-B. Addition of OA+9CLA during differentiation impairs differentiation. (A) Light Microscopy of UN1 iPSC during 4-step cardiac differentiation (methods) supplemented with 100 µM oleic acid and 50 µM 9CLA at different steps. Supplementation with OA+9CLA during differentiation resulted in atypical morphology in the successive steps post 9CLA supplementation. (B) Quantification of portion of complete well death (left) and portion of beating wells resulted by supplementation with 100 µM oleic acid and 50 µM 9CLA at different steps of the 4-step cardiac differentiation (n=24; methods).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DETAILED DESCRIPTION OF THE INVENTION

Dietary intake of linoleic acid in humans is followed by the conjugation of this fatty acid by microbiome bacteria including Bifidobacterium and Lactobacillus to 9CLA which is absorbed and rapidly incorporated into cellular membranes. As embryos develop in a sterile environment in-utero, cells like cardiomyocytes, neurons and beta-cells lack this trans-fat until gut colonialization in the neonatal stage of life. Thus, immature cells lack 9CLA in lipid droplets and cellular membranes. The inability of human cells to conjugate fatty acids means that current stem cell differentiation protocols produce cells that lack 9CLA in their membranes limiting membrane mobility and dramatically effecting cellular bioenergetics. The addition of 9CLA to culture medium can correct this deficiency causing the incorporation of conjugated fatty acids to the cell lipid droplets and phospholipid storage. Agatha et al. Cancer Lett. 2004 (PMID: 15145524) showed that the addition of 40 µM CLA for 48 hours caused cancer cells to accumulate 32 to 63 grams of the conjugated fatty acids for each 100 grams of total phospholipid fatty acid.

Trans fatty acids are known to increase the risk for cardiac and neurovascular damage and thus their intake in infants and adults is recommended to be less than 1% of the daily energy intake according to the Food and Agriculture Organization of the United Nations. In fact, early studies suggested that trans-fat in maternal diet negatively affected birth weight and brain development (PMID: 11724473).

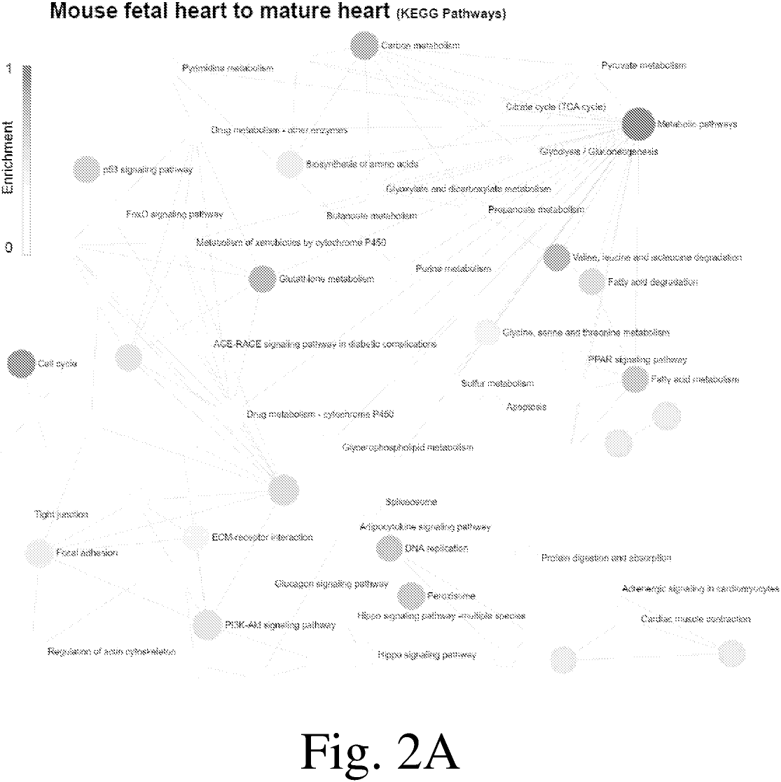

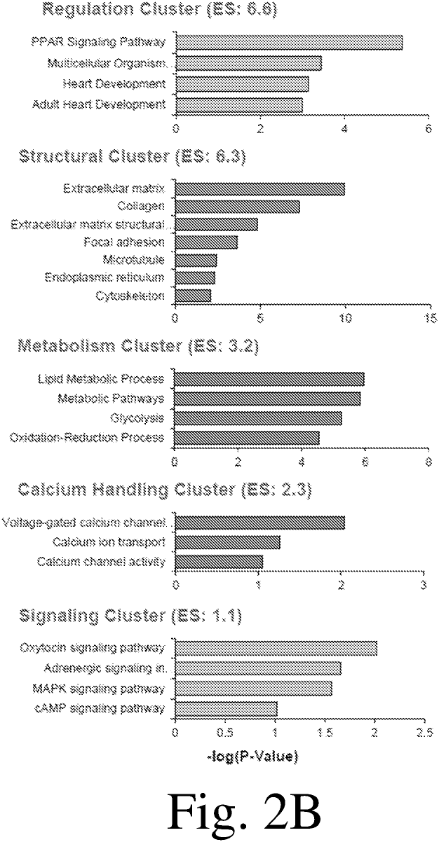

Early work of Raj ala et al. Stem Cell Studies 2011 showed that the addition of conjugated linoleic acid to human embryonic stem cells cultured on mouse fibroblast feeder layers delayed differentiation. Differentiation of stem cell colonies treated with CLA decreased by 20% (FIG. 2B). More recent work by Kennedy et al. J. Nutr. Biochem 2010 on adipogenesis showed that CLA delayed the differentiation of mature adipocytes following short term exposure to CLA, while Larsen et al. the Journal of Nutrition 2010 showed that CLA suppressed myogenic gene expression in human muscle cells, and Taylor et al. Arterioscler Thromb Vasc Biol. 2006 showed a negative effect of CLA on endothelial function in human subjects. In preliminary studies, we generated data showing that addition of CLA during cardiomyocyte differentiation resulted in immature fibroblast-like cells that did not start beating in culture. However, the addition of CLA on terminally differentiated beating cardiomyocytes, mimicking natural post-partum exposure to microbiome derived CLA, resulted in mature cardiomyocytes.

The present invention, in some embodiments thereof, relates to an in vitro method of generating a metabolically mature human cell characterized by an increase in mitochondrial spare capacity as compared to a metabolically immature human cell of the same type. Matured cells exhibit maximal oxygen consumption which is approximately double that observed in basal respiration, thus their spare capacity is equal or greater than basal respiration. More particularly, the invention provides isolated populations of human metabolically mature cells which can be used for a variety of purposes, include for example, screening for compounds affecting toxicity of metabolically mature cardia, pancreatic beta cells and neuronal cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that silencing of the fetal program and mitochondrial maturation is driven in part by peroxisome proliferator-activated receptors (PPAR), induced by the dietary transition to microbiome modified lipid-rich nutrition in the first weeks of life. In fact, such lipid-rich neonatal nutrition could be a missing component needed to drive the final, functional maturation of stem cell-derived parenchyma.

According to an aspect of some embodiments of the invention there is provided an in vitro method of generating a metabolically mature human cell comprising:
(a) culturing a human pluripotent stem cell or a human progenitor cell in the presence of a demethylation promoting agent under conditions suitable for differentiating said human pluripotent stem cell or said human progenitor cell into a metabolically immature cell selected from the group consisting of: a metabolically immature cardiomyocyte, a metabolically immature pancreatic beta cell, and a metabolically immature neuronal cell (e.g., a motor neuron), wherein said human progenitor cell is capable of differentiation into a cardiomyocyte, a pancreatic beta cell, or a neuronal cell, and
(b) culturing said metabolically immature cell resultant of step (a) under culture conditions which increase a mitochondrial spare capacity of said metabolically immature cell by at least 60% above a mitochondrial spare capacity characterizing said metabolically immature cardiomyocyte, said metabolically immature pancreatic beta cell, or said metabolically immature neuron resultant of step (a) as measured by seahorse assay, wherein said culture conditions comprise a culture medium which comprises an effective concentration of a non-conjugated fatty acid selected from the group consisting of: a monounsaturated omega-9 fatty acid, palmitic acid, linoleic acid (LA) and a short chain fatty acid, and an effective concentration of a conjugated fatty acid, thereby generating the metabolically mature human cell.

According to some embodiments of the invention, the culture conditions further increase a basal respiration rate of said metabolically immature cell by at least 30%, at least 40%, at least 50%, at least 60% above a basal respiration rate observed in metabolically immature cardiomyocyte, or said metabolically immature pancreatic beta cell, or said metabolically immature neuron resultant of step (a) as measured a number of methods known in the art, but in preferred embodiments by a “seahorse assay”, described below.

According to some embodiments of the invention, the increase in the mitochondrial spare capacity of the metabolically immature cell is by at least about 65%, about 70%, about 75%, about 80%, about 85%, about 90%, about 95%, 100% or above a basal respiration rate characterizing the metabolically immature cardiomyocyte, the metabolically immature pancreatic beta cell, or the metabolically immature neuron resultant of step (a) as measured by seahorse assay.

As used herein the phrase “basal respiration rate” refers to the oxygen consumption rate of cells in a standard culture medium (without any intervention, e.g., without adding any toxins to the cells and culture medium). See for example, (the world wide web at.agilent.com/cs/library/usermanuals/public/XF_Cell_Mito_Stress_ Test_Kit_User_Guide.pdf) which is incorporated herein by reference and provides detailed instructions for carrying out this assay.

Basal respiration rate is the oxygen consumption used to meet cellular ATP demand resulting from mitochondrial proton leak. The Seahorse XF Mito Stress assay can be used to determine the difference between non-mitochondrial oxygen consumption to the normal untreated oxygen consumption of the cells in units of pmol/min/10,000 cells. Basal respiration in mature cardiomyocytes ranges between 50 to 100 pmol/min/10,000 cells. Basal respiration rates in pancreatic beta-cells ranges between 10 to 20 pmol/min/10,000 cells and ranges between 75 to 120 pmol/min/10,000 cells in matured neuronal cells.

As used herein the “spare capacity” refers to the difference between oxygen consumption rate of cells in standard conditions (basal respiration) and the maximal respiration measured following the injection of an uncoupling agent such as carbonyl cyanide 4-(trifluoromethoxy) phenylhydrazone (FCCP) in a SeaHorse mitoStress Assay. Mitochondrial spare capacity is the capability of the cell to respond to an energy demand. It is calculated using Seahorse XF Mito Stress assay as the difference between the maximal mitochondrial respiration in cells treated with the uncoupling agent and the basal respiration rate of untreated cells. Spare capacity in mature cardiomyocytes ranges between 125 to 225 pmol/min/10,000 cells, while immature cardiomyocytes show spare capacity between 50 to 75 pmol/min/10,000 cells. Spare mitochondrial capacity of metabolically mature pancreatic beta-cells and neurons is about 50%, about 60%, about 70%, about 80%, about 90%, to about 100% higher than basal respiration rates observed in untreated cells.

As used herein, the term “xeno-free media” refers to media that does not contain ingredients derived from non-human animals or recombinant materials made from non-human animal DNA sequences. Such media may also contain purified, processed, or unprocessed materials from human sources.

As used herein the term “about” refers to ± 10 %

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

According to some embodiments of the invention, the human pluripotent stem cell comprises CpG islands of at least 200 nucleotides in nucleic acid sequences of interest which are more than 50% methylated, preferably about 60% methylated.

CpG islands are typically defined as regions with 1) a length greater than 200bp, 2) a G+C content greater than 50%, 3) a ratio of observed to expected CpG greater than 0.6 (Initial sequencing and analysis of the human genome, NATURE | VOL 409 | 15 FEBRUARY 2001, 860-921). Potential CpG islands are identified by searching the draft genome sequence one base at a time, scoring each dinucleotide (+17 for GC, -1 for others) and identifying maximally scoring segments. Each segment was then evaluated to determine GC content (>50%), length (>200) and ratio of observed proportion of GC dinucleotides to the expected proportion on the basis of the GC content of the segment (>0.60), using a modification of a program developed by G. Micklem. WenYin He et al., “Defining Differentially Methylated Regions Specific for the Acquisition of Pluripotency and Maintenance in Human Pluripotent Stem Cells via Microarray”, PLOS One September 2014 https://doi.org/10.1371/journal.pone.0108350.

The methylation state of the DNA (nucleic acid sequence) in a human pluripotent stem cell can be determined by various methods. For example, methylation changes of cells can be determined by using the Human-Methylation 27 K or 250 K BeadChip microarrays ((Illumina) assessing 27,578 unique CpG dinucleotide sites (Simone Bork et al., 2010. “DNA methylation pattern changes upon long-term culture and aging of human mesenchymal stromal cells”. Aging Cell (2010) 9, pp54-63.

As used herein the term “fatty acid” refers to a carboxylic acid with an aliphatic chain.

According to some embodiments of the invention, the aliphatic chain comprises an even number of carbon atoms. For example, the aliphatic chain of the fatty acid can include between 4 to 28 carbon atoms.

The aliphatic compounds can be saturated (saturated fatty acid) joined by single bonds (alkanes), or an unsaturated (unsaturated fatty acid), with double bonds (alkenes) or triple bonds (alkynes). Besides hydrogen, other elements can be bound to the carbon chain, the most common being oxygen, nitrogen, sulfur, and chlorine.

It should be noted that a fatty acid is not a steroid based molecule. Thus, a fatty acid with an aliphatic chain is entirely different from a bile acid such as lithocholic acid, which includes aromatic rings in the backbone.

According to some embodiments of the invention, the conjugated fatty acid is selected from the group consisting of a conjugated linoleic acid which comprises two conjugated double bonds, a conjugated linoleic acid which comprises three conjugated double bonds, 9E,11Z,15E-octadeca-9,11,15-trienoic acid (Rumelenic acid), 9E,11Z,13Z,15E-octadeca-9,11,13,15-tetraenoic acid (α-Parinaric acid), all trans-octadeca-9,11,13,15-tretraenoic acid (β-Parinaric) acid, and 5Z,8Z,10E,12E,14Z-eicosanoic acid (Bosseopentaenoic acid).

Additional known isomers are reviewed in Sheng-hui Wang et al., Poultry Science. 98(10):4632-4639, OCTOBER 2019.

According to some embodiments of the invention, the conjugated linoleic acid which comprises two conjugated double bonds is selected from the group consisting of 9Z,11E-octadeca-9,11-dienoic acid (9CLA, or Rumenic acid or Bovinic acid) and 10E,12Z-octadeca-10,12-dienoic acid (10CLA). In certain embodiments, use of 9CLA in maturation medium is particularly preferred.

According to some embodiments of the invention, the conjugated linoleic acid which comprises three conjugated double bonds is selected from the group consisting of 8E,10E,12Z-octadecatrienoic acid (α-Calendic acid), 8E,10E,12E-octadecatrienoic acid (β-Calendic acid), 8Z,10E,12Z-octadecatrienoic acid (Jacaric acid), 9Z,11E,13E-octadeca-9,11,13-trienoic acid (α-Eleostearic acid), 9E,11E,13E-octadeca-9,11,13-trienoic acid (β-Eleostearic acid), 9Z,11Z,13E-octadeca-9,11,13-trienoic acid (Catalpic acid), and 9Z,11E,13Z-octadeca-9,11,13-trienoic acid (Punicic acid).

As used herein the phrase “short chain fatty acid” refers to a fatty acid characterized by 1-5 carbon atoms in the backbone.

According to some embodiments of the invention the effective concentration of the conjugated fatty acid is capable of activating a peroxisome proliferator-activated receptor (PPAR) nuclear receptor) in the metabolically immature human cell.

PPAR is a member of a subfamily of the nuclear receptor superfamily of transcription factors, plays important roles in lipid and glucose metabolism, and has been implicated in obesity-related metabolic diseases such as hyperlipidemia, insulin resistance, and coronary artery disease.

PPARα (peroxisome proliferator- activated receptor alpha) is a fatty acid-activated member of the PPAR subfamily. It is expressed primarily in metabolic tissues (brown adipose tissue, liver, kidney) but elevated levels are also present in the digestive (jejunum, ileum, colon, gall bladder) and cardiopulmonary (aorta, heart) systems (Sher T, et al. 1993; “cDNA cloning, chromosomal mapping, and functional characterization of the human peroxisome proliferator activated receptor”. Biochemistry 32 5598-604).

PPARγ (peroxisome proliferator- activated receptor gamma) is a fatty acid-activated member of the PPAR subfamily. It is expressed at low levels in most physiological systems, including the central nervous system (CNS), endocrine system, gastrointestinal system, reproductive system, cardiopulmonary system and metabolic tissues, but is most highly expressed in brown and white adipose tissue (Elbrecht A, et al. 1996; “Molecular cloning, expression and characterization of human peroxisome proliferator activated receptors gamma 1 and gamma 2”. Biochem. Biophys. Res. Commun. 224 431-7 V).

Mesoderm progenitor cells are characterized by a positive expression of brachyury (T), mesoderm posterior 1 (MESP1), and NODAL, and a negative expression of SRY (sex determining region Y)-box 1 (SOX1), and SOX2.

Cardiac progenitor cells are characterized by a positive expression of homeobox protein Nkx-2.5 (NKx2.5), kinase insert domain protein receptor (KDR), mesoderm posterior 1 (MESP1), mesoderm posterior 2 (MESP2) and GATA4; and by a negative expression of Myocyte-specific enhancer factor 2C (MEF2C), cardiac troponin T2 (TNNT2) and cardiac troponin I3 (TNNI3).

Adult or Fetal Cardiomyocyte progenitor cells are characterized by a positive expression of Stem cells antigen-1 (Sca-1), Homeobox protein Nkx-2.5 (NKx2.5), Myocyte-specific enhancer factor 2C (MEF2C), GATA4 and GATA6; and by a negative expression of cardiac troponin T2 (TNNT2) and cardiac troponin I3 (TNNI3). Terminally differentiated immature cardiomyocytes are beating cells expressing TNNT2.

Mature cardiomyocytes are characterized by TNNI3 expression, and low expression of TNNI1, Titin isoform N2B (TTN-N2B), Ryanodine Receptor 2 (RYR2), sarco/endoplasmic reticulum Ca2+-ATPase (SERCA2), Gap Junction Protein Alpha 1 (GJA1), and PPARG Coactivator 1 Alpha (PPARGC1A). Mature cardiomyocytes have CLA incorporated in their membranes changing cholesterol affinity and membrane mobility.

Pancreatic progenitor cells or adult pancreatic stem cells are characterized by expression of homeobox protein Nkx-2.2 (NKX2.2), NKX6.1, and basic helix-loop-helix protein PTF1A (P48). Terminally differentiated immature pancreatic beta-cells are insulin-secreting cells expressing NKX6.1.

Mature pancreatic beta cells express IAPP, HOPX, NEFM, SIX2, UCN3, MAFA and SIX3 downregulated immature gene markers including LDHA and IGF2. Mature pancreatic beta-cells have CLA incorporated in their membranes changing cholesterol affinity and membrane mobility.

Mesenchymal stem cells (MSC) are characterized by a positive expression of cluster of differentiation 44 (CD44), CD90, CD105, CD106, CD166, and Stro-1; and a negative expression of CD14 and CD34.

Neural ectoderm cell is characterized by a positive expression of SOX2 and orthodenticle homeobox 2 (OTX2).

Fetal or adult neural stem cell is characterized by a positive expression of Nestin and SOX2.

Terminally differentiated immature neurons are cells that create active action potential upon electrical induction expressing Nestin.

Mature neuronal markers express Hexaribonucleotide Binding Protein-3 (NeuN), Microtubule-associated protein 2 (MAP2) and Synaptophysin (SYP) as well as neuron type-specific markers that include Astrocytes markers ALDH1L1, ALDOC, CD44, EAAT1, and EEAT2. Midbrain dopaminergic maturation markers Calbindin, DAT, GIRK2, TH, and vDAT. Forebrain cholinergic maturation markers include: ChAT, vAChT, MAP2, p75NTR, and TrKA. Oligodendrocytes maturation markers Olig2, MBP, MOG, and SOX10. Motor neurons maturation markers include Olig2, Islet1, Islet2, and Neuroginen. Mature neurons have CLA incorporated in their membranes changing cholesterol affinity and membrane mobility.

According to some embodiments of the invention, the toxicological end-point assay is a Cell Viability Assay (e.g., Live/Dead Assay), Functional Viability Assay (e.g., MTT), Calcium Handling Assay (e.g., Flu-4), Inflammation/Injury Marker Assay (e.g., ELISA for IL-10) or any other standard assays published by TOX21, EuroTOX, EPA or any other governmental.

According to an aspect of some embodiments of the invention, there is provided a method of selecting a compound which is toxic to cells, comprising:
(a) incubating the isolated population of cells of some embodiments of the invention with the compound for a pre-determined time period, and;
(b) measuring following said pre-determined time period a level of metabolic activity selected from the group consisting of: an intracellular esterase activity and a conversion of MTT 3-(4,5-dimethylthiazol-2-yl)-2,5-diphenyltetrazolium bromide to insoluble formazan, wherein a decrease in said level below 90% indicates that said compound is toxic to the cells,
thereby selecting a compound which is toxic to cells.

According to some embodiments of the invention, the intracellular esterase activity is measured in living cells.

According to some embodiments of the invention, the intracellular esterase activity is measured by the enzymatic conversion of non-fluorescent calcein AM to the fluorescent calcein.

According to some embodiments of the invention, the conversion of MTT 3-(4,5-dimethylthiazol-2-yl)-2,5-diphenyltetrazolium bromide to its insoluble formazan results in a purple color.

Table 1 provides a number of demethylating promoting agents that can be used to advantage in protocols for differentiation of stem cells or iPSCs into metabolically immature differentiated cells of interest.

TABLE 1

Demethylation promoting agents

| | |
|---|---|
| Type/pathway | Exemplary agents |
| GSK inhibitor | CHIR99021 |
| | CHIR 98014 |
| | Lithium ion |
| | Valproic acid |
| | Iodotubercidin |
| | Naproxen |
| | Cromolyn |
| | Famotidine |
| | Curcumin |
| | Olanzapine |
| | Pyrimidine and Furopyrimidine Derivatives (e.g [1-aryl-1H-pyrazolo[3,4-d]pyrimidin-4-yl]arylhydrazones) |
| | Maleimide Derivatives (e.g. 3-anilino-4-arylmaleimides ) |
| | Staurosporine |
| | Organometallic Inhibitors |
| | Indole Derivatives (e.g indirubin-3′-monoxime) |
| | Paullone Derivatives (e.g 9-nitro-paullone) |
| | Pyrazolamide Derivatives |
| | Oxadiazole Derivatives |
| Type/pathway | Exemplary agents |
| | Thiazole Derivatives (e.g AR-A014418) |
| MEK Inhibitor | PD0325901 |
| | TAK-733 |
| | CI-1040 |
| | Selumetinib |
| | Binimetinib (MEK162) |
| | Cobimetinib (XL518) |
| | Trametinib (GSK1120212) |
| Cofactors for chromatin-modifying enzymes (TET) | Alpha-ketoglutarate |
| DNA methyltransferases inhibitor | Azacitidine |
| | Decitabine |
| | Zebularine |
| | CP-4200 |
| | SGI-110 |
| | MG98 |
| | RG108 |
| | Procainamide |
| Natural methyltransferases inhibitor | Epigallocatechin-3-gallate |
| | laccaic acid A |

TABLE 2

Composition of breast milk from various mammals

| Component (g/100g milk) | Mouse | Goat | Sheep | Cow | Marmoset | Macaque | Human |
|---|---|---|---|---|---|---|---|
| Protein | 9.0 | 2.9 | 6.0 | 3.2 | 3.6 | 1.3 | 1.3 |
| Carbohydrate | 3.0 | 4.7 | 5.4 | 4.6 | 7.5 | 9.2 | 7.1 |
| Fat | 13.1 | 3.8 | 7.0 | 3.7 | 7.7 | 3.3 | 4.6 |

Fat Composition (% of total fat)

| | | | | | | | |
|---|---|---|---|---|---|---|---|
| Saturated fat | 38.8% | 64.7% | 65.8% | 57.4% | 67.4% | 51.9% | 47.8% |
| Monounsaturated fat | 38.6% | 26.8% | 24.6% | 25.0% | 25.6% | 36.3% | 40.0% |
| Polyunsaturated fat | 17.4% | 3.6% | 4.4% | 6.0% | 6.9% | 11.6% | 11.7% |
| Omega-3 fatty acids | 2.6% | 1.1% | 3.9% | 2.0% | 3.1% | 3.0% | 1.4% |
| Omega-6 fatty acids | 14.8% | 2.9% | 2.8% | 3.2% | 3.9% | 8.6% | 11.5% |
| Cholesterol | 0.1% | 0.3% | 0.4% | 0.3% | 0.1% | 0.1% | 0.3% |
| Other FAs (CLA, PA, etc.) | 5.1% | 4.7% | 4.8% | 11.3% | 0.1% | 0.1% | 0.1% |

Table 2. Breast milk composition of mammalian species. Breast milk of mouse, goat, sheep, cow, non-primate monkey (marmoset), primates (macaque) and human breast milk. Adapted from Turton et al. 1978, Whittier et al. 2011, Oosting et al, 2015, USDA Nutrient Database (2019), Milligan et al. 2007.

TABLE 3

Fatty acid composition of breast milk in human and mouse

| % of total fatty acids (w/w) | Fatty Acid (FA) | Human | Mouse |
|---|---|---|---|
| De novo FAs | Caprylic (C8:0) | 0.1 ±0.1 | 0.4 ±0.1 |
| | Capric (C10:0) *** | 1.1 ±0.3 | 5.8 ±0.5 |
| | Hendecanoic (C11:0) *** | 0 | 0.4 ±0.1 |
| | Lauric (C12:0) * | 5.9 ±0.8 | 10.0 ±0.7 |
| | Myristic (C14:0) | 7.9 ±1.0 | 12.3 ±0.6 |
| | Myristoleic (C14:1 n-5) ** | 0 | 0.2 ±0.1 |
| Preformed FAs | Palmitic (C16:0) | 23.9 ±0.5 | 23.7 ±0.9 |
| | Palmitoleic (C16:1 n-7) | 2.3 ±0.4 | 3.0 ±0.2 |
| | trans-Palmitoleic (C16:1t n-7) *** | 0 | 0.25 ±0.1 |
| | Stearic (C18:0) *** | 7.4 ±0.6 | 1.7 ±0.1 |
| | Oleic (C18:1 n-9) *** | 35.8 ±0.7 | 21.3 ±1.2 |
| | Vaccenic (C18:1) *** | 0 | 3.1 ±0.2 |
| | Arachidic (C20:0) | 0.5 ±0.2 | 0 |
| | Gadoleic (C20:1 n-9) | 0.3 ±0.1 | 0.7 ±0.1 |
| | Eicosadienoic (C20:2) *** | 0 | 1.1 ±0.1 |
| | Dihomo-γ-linolenic (C20:3 n-6) | 0.3 ±0.1 | 0.6 ±0.1 |
| Essential FAs | Linoleic (C18:2 n-6) | 10.9 ±0.9 | 14.1 ±0.4 |
| | α-linolenic (C18:3 n-3) | 1.0 ±0.4 | 1.2 ±0.1 |
| Conditionally Essential FAs | Arachidonic (C20:4 n-6) | 0.4 ±0.1 | 0.5 ±0.1 |
| | DHA (C22:6 n-3) | 0.4 ±0.3 | 0 |

Table 3. Fatty acid composition of human and mouse breast milk. Adapted from Gardner et al, 2017 and Saben et al, 2014. Values are presented as % of total fatty acids ± standard error. Significant differences between human and mice are noted as * p<0.05; *** p<0.01; *** p<0.001

The tables below provide specific protocols and reagents that can be used to advantage to differentiate stem cells into cardiomyocytes, pancreatic beta cells and neuronal cells.

TABLE 4

Exemplary protocol for differentiation of human pluripotent stem cells into metabolically mature cardiomyocytes

| Stage | | Days | Media Components | Notes |
|---|---|---|---|---|
| S0 | | 0 | mTeSR1/mTESR Plus | Once the cells are at ~70% confluence, passage using Accutase. |
| S1 | Primitive Streak Mesoderm Induction | 1-2 | Car-S1 medium: CDM3 [RPMI, Recombinant Human Albumin (0.5 mg/ml), L-Ascorbic acid 2-phosphate (0.25 mg/ml), Pen-Penicillin (100 U), Streptomycin (100 µg/ml)], CHIR99021 (6 µM) | Differentiation should start at 50-60% confluence. Cells are washed twice with PBS and S1 medium is added. Daily media change |
| S2 | Cardiac Mesoderm Specification | 3-4 | Car-S2 medium: CDM3 [RPMI, Recombinant Human Albumin (0.5 mg/ml), L-Ascorbic acid 2-phosphate (0.25 mg/ml), Pen-Penicillin (100 U), Streptomycin (100 µg/ml)], Wnt-C59 (2 µM) | On day 3, Cells are washed twice with PBS before switching to S2 medium. Daily media change |
| S3 | Cardiac Differentiation | 5-10 | Car-S3 medium: RPMI, Penicillin (100 U), Streptomycin (100 µg/ml), B27 supplement minus insulin (1X). | On day 5 the cells are washed twice with PBS before switching to S3 medium. Daily media change |
| S4 | Cardiac Maturation | 11-15 | Car-S4 medium: RPMI, Penicillin (100 U), Streptomycin (100 µg/ml), B27 supplement minus insulin (1X), OA (100 µM), 9CLA (50 µM). | On day 11, Cells are washed twice with PBS before switching to S4 medium. Daily media change |

TABLE 5

Exemplary protocol for differentiation of methylated human pluripotent stem cells into metabolically mature cardiomyocytes

| Stage | | Days | Media Components | Notes |
|---|---|---|---|---|
| S0 | Demethylation | -1-0 | mTeSR1 supplemented with CHIR99021 (6 µM) | Once the cells are at ~70% confluence, passage using Accutase. |
| S1 | Primitive Streak Mesoderm Induction | 1-2 | Car-S1 medium: CDM3 [RPMI, Recombinant Human Albumin (0.5 mg/ml), L-Ascorbic acid 2-phosphate (0.25 mg/ml), Pen-Penicillin (100 U), Streptomycin (100 µg/ml)], CHIR99021 (6 µM) | Differentiation should start at 50-60% confluence. Cells are washed twice with PBS and S1 medium is added. Daily media change |
| S2 | Cardiac Mesoderm Specification | 3-4 | Car-S2 medium: CDM3 [RPMI, Recombinant Human Albumin (0.5 mg/ml), L-Ascorbic acid 2-phosphate (0.25 mg/ml), Pen-Penicillin (100 U), Streptomycin (100 µg/ml)], Wnt-C59 (2 µM) | On day 3, Cells are washed twice with PBS before switching to S2 medium. Daily media change |
| S3 | Cardiac Differentiation | 5-10 | Car-S3 medium: RPMI, Penicillin (100 U), Streptomycin (100 µg/ml), B27 supplement minus insulin (1X). | On day 5 the cells are washed twice with PBS before switching to S3 medium. Daily media change |
| S4 | Cardiac Maturation | 11-15 | Car-S4 medium: RPMI, Penicillin (100 U), Streptomycin (100 µg/ml), B27 supplement minus insulin (1X), OA (100 µM), 9CLA (50 µM). | On day 11, Cells are washed twice with PBS before switching to S4 medium. Daily media change |

TABLE 6

Materials for hPSC cardiac differentiation into metabolically mature cardiomyocytes

| Materials for hPSC cardiac differentiation | Company | Catalog # |
|---|---|---|
| mTeSR | STEM CELL Technologies | 5850 |
| Cultrex PathClear Reduced Growth Factor Basement Membrane Extracts | Trevigen | 3433-005-01 |
| Thiazovivin | Cayman | 14245 |
| TrypLE Express | Life Technologies | 12604-013 |
| 0.5 M EDTA | Invitrogen | 15575-020 |
| DNase I, Bovine Pancreas | Biological Industries | 260913-10MU |
| CDM3 Medium | | |
| RPMI 1640 Medium | Gibco | 21875-034 |
| Recombinant Human Albumin | Sigma-Aldrich | A9731 |
| L-Ascorbic acid 2-phosphate | Sigma-Aldrich | A8960 |
| Pen-Strep Solution | Biological Industries | 03-031-1B |
| Differentiation Reagents | | |
| CHIR99021 | Stemgent | 04-0004-10 |
| Wnt-C59 | selleckchem | S7037 |
| RPMI-B27 Medium | | |
| RPMI 1640 Medium | Gibco | 21875-034 |
| B27 supplement minus insulin | Life Technologies | A18956-01 |
| Pen-Strep Solution | Biological Industries | 03-031-1B |
| Oleic acid - albumin | Sigma | O3008 |
| 9-cis, 11-trans, conjugated linoleic acid (9CLA) | Sigma | 16413 |
| Albumin Bovine (BSA) Fraction V | MP | 160069 |

Table 6. List of materials for hPSC cardiac differentiation.

TABLE 7

An exemplary protocol for pancreatic beta cell differentiation into metabolically mature pancreatic beta cells

| Stage | | Days | Media Components |
|---|---|---|---|
| 0 | | 0 | mTeSR1/mTESR Plus |
| 1 | Definitive Endoderm Induction | 1 | Pan-S1, 100 ng/ml ActivinA, 3 µM Chir99021 |
| | | 2 | Pan-S1, 100 ng/ml ActivinA |
| 2 | Primitive Gut Tube Specification | 46 | Pan-S2, 50 ng/ml KGF |
| 3 | | 7 | Pan-3, 50 ng/ml KGF, 0.25 µM Sant1, 2 µM RA, 200 nM LDN193189, 500 nM PdBU |
| | Posterior Foregut Specification | 8 | Pan-S3, 50 ng/ml KGF, 0.25 µM Sant1, 2 µM RA, 500 nM PdBU |
| 4 | Pancreatic Endoderm Specification | 9 11 13 | Pan-S3, 50 ng/ml KGF, 0.25 µM Sant1, 100 nM RA |
| 5 | Endocrine Progenitors Specification | 14 16 | Pan-S5, 0.25 µM Sant1, 100 nM RA, 1 µM XXI, 10 µM Alk5i II, 1 µM T3, 20 ng/ml Betacellulin |
| 6 | Pancreatic Progenitors Specification | 18 20 | Pan-S5, 25 nM RA, 1 µM XXI, 10 µM Alk5i II, 1 µM T3, 20 ng/ml Betacellulin |
| 7 | Beta-Cells Differentiation | 21-28 (Every 2) | Pan-S6, 10 µM Alk5i II, 1 µM T3 |
| 8 | Beta-Cells Maturation | 29-35 (Every 2) | Pan-S6, 10 µM Alk5i II, 1 µM T3, OA (100 µM), 9CLA (50 µM). |

TABLE 8

An exemplary protocol for methylated pancreatic beta cell differentiation into metabolically mature pancreatic beta cells

| Stage | | Days | Media Components |
|---|---|---|---|
| 0 | Demethylation | -1-0 | mTeSR1 supplemented with 3 µM Chir99021 |
| 1 | Definitive Endoderm Induction | 1 | Pan-S1, 100 ng/ml ActivinA, 3 µM Chir99021 |
| | | 2 | Pan-S1, 100 ng/ml ActivinA |
| 2 | Primitive Gut Tube Specification | 46 | Pan-S2, 50 ng/ml KGF |
| 3 | Posterior Foregut Specification | 7 | Pan-S3, 50 ng/ml KGF, 0.25 µM Sant1, 2 µM RA, 200 nM LDN193189, 500 nM PdBU |
| | | 8 | Pan-S3, 50 ng/ml KGF, 0.25 µM Sant1, 2 µM RA, 500 nM PdBU |
| 4 | Pancreatic Endoderm Specification | 9 11 13 | Pan-S3, 50 ng/ml KGF, 0.25 µM Sant1, 100 nM RA |
| 5 | Endocrine Progenitors Specification | 14 16 | Pan-S5, 0.25 µM Sant1, 100 nM RA, 1 µM XXI, 10 µM Alk5i II, 1 µM T3, 20 ng/ml Betacellulin |
| 6 | Pancreatic Progenitors Specification | 18 20 | Pan-S5, 25 nM RA, 1 µM XXI, 10 µM Alk5i II, 1 µM T3, 20 ng/ml Betacellulin |
| 7 | Beta-Cells Differentiation | 21-28 (Every 2) | Pan-S6, 10 µM Alk5i II, 1 µM T3 |
| 8 | Beta-Cells Maturation | 29-35 (Every 2) | Pan-S6, 10 µM Alk5i II, 1 µM T3, OA (100 µM), 9CLA (50 µM). |

Following are the compositions of the culture media described herein for pancreatic beta cell differentiation and maturation:

Pan-S1 media: MCDB131 (Cellgro; 15-100-CV) + 8 mM D-(+)-Glucose (Sigma; G7528) + 2.46 g/L NaHCO₃ (Sigma; S3817) + 2 % FAF-BSA (Proliant; 68700) + ITS-X (Invitrogen; 51500056) 1:50.000 + 2 mM Glutamax (Invitrogen; 35050079) + 0.25 mM Vitamin C (Sigma Aldrich; A4544) + 1 % Pen/Strep (Cellgro; 30-002-CI).

Pan-S2 media: MCDB131 + 8 mM D-Glucose + 1.23 g/L NaHCO3 + 2 % FAF-BSA + ITS-X 1:50.000 + 2 mM Glutamax + 0.25 mM Vitamin C + 1% Pen/Strep.

Pan-S3 media: MCDB131 + 8 mM D-Glucose + 1.23 g/L NaHCO₃ + 2% FAF-BSA + ITS-X 1:200 + 2 mM Glutamax + 0.25 mM Vitamin C + 1% Pen/Strep.

Pan-S5 media: MCDB131 + 20 mM D-Glucose + 1.754 g/L NaHCO₃ + 2% FAF-BSA + ITS-X 1:200 + 2 mM Glutamax + 0.25 mM Vitamin C + 1% Pen/Strep + Heparin 10 µg/ml (Sigma; H3149).

Pan-S6 media: CMRL 1066 Supplemented + 10% FBS + 1% Pen/Strep.

TABLE 9

Reagents useful for hPSC pancreatic beta cells differentiation and available commercial sources

| | |
|---|---|
| Materials for hPSC pancreatic beta cells differentiation | Company |
| mTeSR | STEM CELL Technologies |
| Matrigel Reduced Growth Factor Basement Membrane Matrix | Coming |
| Thiazovivin | Cayman |
| TrypLE Express | Life Technologies |
| Ethylenediaminetetraacetic acid (EDTA) | Invitrogen |
| DNase I, Bovine Pancreas | Biological Industries |
| MCDB131 | Cellgro |
| 8 mM D-(+)-Glucose | Sigma |
| Sodium bicarbonate (NaHCO3) | Sigma |
| Bovine Serum Albumin fatty acid free (FAF-BSA) | Proliant |
| Insulin-Transferrin-Selenium-Ethanolamine (ITS-X) | Invitrogen |
| Glutamax | Invitrogen |
| Vitamin C | Sigma |
| Pen-Strep Solution | Biological Industries |
| Heparin | Sigma |
| CMRL 1066 Supplemented | Mediatech |
| Fetal bovine serum (FBS) | Biological Industries |
| CHIR99021 | Stemgent |
| ActivinA | R&D Systems |
| Keratinocyte growth factor (FGF7) | Peprotech |
| Sant1 | Sigma |
| Retinoic acid (RA) | Sigma |
| LDN193189 | Sigma |
| Phorbol 12,13-dibutyrate (PdBU) | EMD Millipore |
| γ-Secretase Inhibitor XXI (XXI) | EMD Millipore |
| ALK5 Inhibitor II (Alk5i II) | Axxora |
| Triiodothyronine (T3) | EMD Millipore |
| Betacellulin | Thermo Fisher Scientific |
| Oleic acid - albumin | Sigma |
| 9-cis, 11-trans, conjugated linoleic acid (9CLA) | Sigma |
| Albumin Bovine (BSA) Fraction V | MP |

TABLE 10

Exemplary protocol for human pluripotent stem cells differentiation into metabolically mature motor neurons

| Stage | | Days | Media Components |
|---|---|---|---|
| S0 | | 0 | mTeSR1/mTESR Plus |
| S1 | | 1 | Dissociate Cells, S1 Media, 1% RevitaCell. |
| | Neural Ectoderm Induction | 2-7 | Neu-S1 Media |
| S2 | Neuron Progenitor Specification | 8-13 | Neu-S2 Media, 10 ng/mL bFGF and 10 ng/mL EGF. |
| S3 | Motor Neuron Specification | 16 | Dissociate Cells, Neu-S2 Media, 10 µM all-trans retinoic acid, 100 ng/ml rSHH, 1 µM Purmorphamine and 1 mM SAG Dihydrochloride. |
| | | 17-22 | Neu-S2 Media, 10 µM all-trans retinoic acid, 100 ng/ml rSHH, 1 µM Purmorphamine and 1 mM SAG Dihydrochloride. |
| S4 | Motor Neuron Differentiation | 23 | Dissociate Cells, Neu-S2 Media, 10 ng/mL CNTF, 10 ng/ml BDNF, 10 ng/mL NT-3 and 10 ng/mL GDNF. |
| | | 24-29 | Neu-S2 Media, 10 ng/mL CNTF, 10 ng/ml BDNF, 10 ng/mL NT-3 and 10 ng/mL GDNF. |
| S5 | Motor Neuron Maturation | 29-33 | Neu-S3 Media |

TABLE 11

Exemplary protocol for methylated human pluripotent stem cells differentiation into metabolically mature motor neurons

| Stage | | Days | Media Components |
|---|---|---|---|
| S0 | Demethylation | -1-0 | mTeSR1 supplemented with 3 µM CHIR99021 |
| S1 | Neural Ectoderm Induction | 1 | Dissociate Cells, Neu-S1 Media, 1% RevitaCell. |
| | | 2-7 | Neu-S1 Media |
| S2 | Neuron Progenitor Specification | 8-13 | Neu-S2 Media, 10 ng/mL bFGF and 10 ng/mL EGF. |
| S3 | Motor Neuron Specification | 16 | Dissociate Cells, Neu-S2 Media, 10 µM all-trans retinoic acid, 100 ng/ml rSHH, 1 µM Purmorphamine and 1 mM SAG Dihydrochloride. |
| | | 17-22 | Neu-S2 Media, 10 µM all-trans retinoic acid, 100 ng/ml rSHH, 1 µM Purmorphamine and 1 mM SAG Dihydrochloride. |
| S4 | Motor Neuron Differentiation | 23 | Dissociate Cells, Neu-S2 Media, 10 ng/mL CNTF, 10 ng/ml BDNF, 10 ng/mL NT-3 and 10 ng/mL GDNF. |
| | | 24-29 | Neu-S2 Media, 10 ng/mL CNTF, 10 ng/ml BDNF, 10 ng/mL NT-3 and 10 ng/mL GDNF. |
| S5 | Motor Neuron Maturation | 29-33 | Neu-S3 Media |

Following are the compositions of the culture media described herein for neuronal differentiation and maturation:

Neu-S1 medium: Neural induction medium (NIM) 1:1 mix of KO-DMEM/F:12 and NBM, 10% KSR, 1% NEAA, 1% GlutaMAX, 0.1 mM L-AA, 2 µM SB431542, 3 µM CHIR99021, 1 µM dorso-morphin and 1 µM compound E.

Neu-S2 medium: NPC expansion medium (NEM).1:1 mix of KO-DMEM:F12 and NBM, 1% P/S, 1% B27, 1% N2, 1% NEAA, 1% GlutaMAX, 0.1 mM L-AA,

Neu-S3 medium: MN maturation medium. 1:1 KO-DMEM:F12 and NBM, 1% P/S, 1%N2, 1% NEAA, 1% GlutaMAX, 0.1 mM L-AA, 10 ng/mL CNTF, 10 ng/ml BDNF, 10 ng/mL NT-3 and 10 ng/mL GDNF, OA (100 µM), 9CLA (50 µM).

Dissociate Cells refer to dissociation using accutase and plating at 0.5 × 10⁶ cells/well on Matrigel(GFR)-coated plates.

The following examples are provided to illustrate certain embodiments of the invention. They are not intended to limit the invention in any way.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

### Human Subjects

All protocols involving human tissue were reviewed and exempted by the Hebrew University of Jerusalem and Weill Cornell Medical College Institutional Review Boards.

### Mice

C57B⅙ mice were purchased from Harlan and allowed to acclimatize for 2 weeks before experimentation. GF mice were bred at the Weizmann Institute germ-free facility. In all experiments, age-matched mice were used as indicated in the relevant section. Postnatal maturation of the liver was examined in 4 weeks old GF or SPF mice. All experimental procedures were approved by the local IACUC (IACUC application no. 10320119-2).

### Cells and Cell Culture

Human embryonic stem cells lines I3, HuES-8, H9 and H1 and induced pluripotent stem cells lines UN-1 were cultured on growth factor reduced Matrigel (BD Biosceinces, San Jose, CA) in mTeSR-1 media (StemCell Technologies, Vancouver, Canada). Cells were obtained from the Technion (Prof. J. Itskovitz-Eldor), the Harvard Stem Cell Institute (Boston, MA), and WiCell (Madison, WI), respectively, authenticated at source and tested for mycoplasma contamination using PCR. Cells were serially passaged using Accutase (Merck, USA) and grown in a humidified incubator at 37° C. and 5% CO₂. Each of these cell types can be used as starting cells for generation of metabolically mature cardiomycytes, pancreatic beta cells or neuronal cells.

Cryopreserved human hepatocytes were purchased from XenoTech (Lenexa, KS), Lonza (Switzerland) or Life Technologies (Grand Island, NY), thawed and plated on growth factor reduced Matrigel in Hepatocyte Maintenance Medium (Lonza, Germany) according to manufacturer instructions.

### Methods

### GeneChip Analysis

Conventionalized-WT, germ-free-WT, and Conventionalized-Pparα^{-/-} mice heart GeneChip (Affymetrix Mouse Genome 430 2.0 Array) data were downloaded from GEO database accession GSE14929, along with accompanying metadata. Fetal and adult mice hearts GeneChip (Illumina MouseRef-8 v2.0 expression beadchip) data were downloaded from GEO database accession GSE129090, along with accompanying metadata. Expression levels of all genes were quantified using GEO2R. Expression matrix of inferred gene counts and a differential expression analysis was performed using GEO2R and LIMMA using default parameters. Reported are p-values from a negative binomial Wald-test. Overall, the present inventors found 1087 genes that showed significant differential mRNA expression between the conventionalized-WT and germ-free-WT hearts with a BH-FDR<0.04 and p<0.002.

### Processing, Analysis, and Graphic Display of Genomic Data

R studio (https://www(dot)rstudio(dot)com/) was used to perform principle component analysis (PCA; prcomp package), scatter plots and volcano plots (ggplot package). Hierarchical clustering, heat maps, correlation plots and similarity matrixes were made in Morpheus. Gene ontology enrichment analyses and clustering were performed using DAVID Informatics Resources 6.7. Network maps were with the McGill’s Network Analyst Tool using the KEGG database.

### Quantitative Real-Time PCR (qRT-PCR)

Total RNA was extracted and isolated from homogenized tissue specimen or cell pellets using NucleoSpin RNA II kit (Macherey-Nagel, Germany) according to the manufacturer’s instructions. RNA concentration and purity were determined using NanoDrop ND-1000 spectrophotometer (Thermo Fisher Scientific, USA). cDNA synthesis was performed using qScript™ cDNA Synthesis (Quanta BioSciences) according to the manufacturer’s instructions. 1 µg of purified RNA was used for each reaction, with concentration and purity determined by an ND-1000 spectrophotometer (NanoDrop Technologies). Each reaction was diluted to reach a concentration of 10 ng/µL. mRNA expression levels were measured by qRT-PCR using KAPA SYBR FAST (Kapa Biosystems) on Applied Biosystems™ QuantStudio™ 5 Real-Time PCR System. Gene transcription was evaluated using the ΔΔCt method normalized to ribosomal protein L32 (RPL32) and ubiquitin-conjugating enzyme (UBC1). Primer sequences and sources are listed in Table 12 below.

TABLE 12

Primers used for human sequences

| Gene Name | Forward 5′-3′ | SEQ ID NO: | Reverse 5′-3′ | SEQ ID NO: |
|---|---|---|---|---|
| ABCG5 | TCTGTTTCCCGTGCTGCGAG | 1 | CCCAGCGTCCAGTAGCACAC | 23 |
| CPT1α | GCCTCGTATGTGAGGCAAA | 2 | CCCATTCGTAGCCTTTGGTA | 24 |
| FIS1 | AAAGTACGTCCGCGGGTTGC | 3 | TCCGATGAGTCCGGCCAGT | 25 |
| MFN2 | AAGGTGAAGCGCAATGTCCCT | 4 | CCCCCAGCTGCTCAAAAATGC | 26 |
| PGC1α | TGCTCTGTGTCACTGTGGATTG G | 5 | GGGCAAAGAGGCTGGTCTTCA | 27 |
| PPARα | | Purchased from Qiagen (QT00017451) | | |
| OCT4 | TCTCCAGGTTGCCTCTCACT | 6 | GTGGAGGAAGCTGACAACAA | 28 |
| SERPINA 1 | ACGAGACAGAAGACGGCATT | 7 | CCCTCTGGATCCACTGCTT | 29 |
| SOX2 | GCTTAGCCTCGTCGATGAAC | 8 | AACCCCAAGATGCACAACTC | 30 |
| MAPK1 | TCTGGAGCAGTATTACGACCC | 9 | CTGGCTGGAATCTAGCAGTCT | 31 |
| PPP1R1A | TCTCAGGCATACGCTGACTAC | 10 | GGAGACTCTGTACTCGAAGGT | 32 |
| NPPA | CAACGCAGACCTGATGGATTT | 11 | AGCCCCCGCTTCTTCATTC | 33 |
| NPPB | TGGAAACGTCCGGGTTACAG | 12 | CTGATCCGGTCCATCTTCCT | 34 |
| TNNT2 (cTnT) | ACAGAGCGGAAAAGTGGGAA G | 13 | TCGTTGATCCTGTTTCGGAGA | 35 |
| TNNI3 (cTnI) | CGTGTGGACAAGGTGGATGAA G | 14 | GCCGCTTAAACTTGCCTCGAA G | 36 |
| DGS2 | GAAGAGTTGAGTGCAGCACAT AC | 15 | CTTGCTTCTACTGTCAAAGTCT ACG | 37 |
| PKP2 | TTTAATATTTGCTTGATTGCAG | 16 | TGTCATAGGTTTTAGGAACAG G | 38 |
| α1A | GGCTCCTTCTACCTGCCTCT | 17 | AGGGCTTGAAATCAGGGAAG | 39 |
| α1B | CCTGAGGATCCATTCCAAGA | 18 | GGTTGAGGCAGCTGTTGAAG | 40 |
| SCN4B | TCCTGCGCTATGAGAATGAGG | 19 | TGGTGTTGTGCTCGTAGTTTTC | 41 |
| KCNE1 | TGTGGCAGGAGACAGTTCAG | 20 | GCTTCTTGGAGCGGATGTAG | 42 |
| RPL32 | CTCTTTCCACGATGGCTTTG | 21 | GTCAAGGAGCTGGAAGTGCT | 43 |
| UBC | CGGGTGTGGCACAGCTAGTT | 22 | TGCATTGTCAAGTGACGATCA C | 44 |

### Transmission Electron Microscopy (TEM)

For TEM analysis, cells were seeded in a plastic 8 chamber slide (Lab-Tek) and fixed in 2.5% Glutaraldeyde, 2% paraformaldehyde in 0.1 M Cacodylate buffer composed of 0.1 M cacodylic acid Na(CH₃)₂AsO₂H balanced with NaOH to pH of 7.4, for 2 hours at room temp and incubated at 40° C. overnight. Cells were then rinsed 4 times, 10 minutes each, in cacodylate buffer and post fixed and stained with 1% osmium tetroxide, 1.5% potassium ferricyanide in 0.1 M cacodylate buffer for 1 hour. Cells were then washed 4 times in cacodylate buffer followed by dehydration in increasing concentrations of ethanol consisting of 30%, 50%, 70%, 80%, 90%, 95%, for 10 minutes each step followed by 100% anhydrous ethanol 3 times, 20 minutes each. Following dehydration, the cells were infiltrated with increasing concentrations of Agar 100 resin in ethanol, consisting of 25, 50, 75, and 100% resin for 16 hours each step. The cells then were embedded in fresh resin and let polymerize in an oven at 600° C. for 48 hours.

Embedded cells in blocks were sectioned with a diamond knife on an LKB 3 microtome and ultrathin sections (80 nm) were collected onto 200 Mesh, thin bar copper grids. The sections on grids were sequentially stained with Uranyl acetate and Lead citrate for 10 minutes each and viewed with Tecnai 12 TEM 100 kV (Phillips, The Netherlands) equipped with MegaView II CCD camera. Mitochondria diameter and cell/nuclei size were measured manually using Analysis® version 3.0 software (SoftImaging System GmbH, Germany).

### Immunofluorescence Microscopy

Cultured cells were fixed using 4% paraformaldehyde for 15 minutes at room temperature. Cells were then permeabilized in blocking solution (2% bovine serum albumin (BSA) and 0.25% Triton X-100 in PBS) for one hour at room temperature and incubated with primary antibodies (Table 13 below) for an additional hour. Following washes, cells were incubated with secondary antibodies (Table 14 below) for 1 hour at room temperature in blocking solution, washed twice in PBS and counterstained with 1 µg/ml Hoechest 33258 (Sigma Aldrich, USA) for 5 minutes. Imaging was performed on a Zeiss LSM 700 confocal microscope. Staining of actin was carried out simultaneously with secondary antibody incubation step by adding a 1:100 of Rhodamine-Phalloidin solution. Microscopic single cell and mitochondrial shape analysis was carried out using the CellProfiler software. Mitochondrial elongation was scored from 0 to 1, where 0 corresponds to a circle and 1 to a rectangle.

TABLE 13

Primary antibodies, source and dilutions

| Antibody | Host | Company | Catalog Number | Dilution |
|---|---|---|---|---|
| HSP60 (k-19) | Goat | Santa Cruz | SC-1722 | 1:100 |
| OCT¾ | Rabbit | Santa Cruz | SC9081 | 1:100 |
| TNNI3 | Rabbit | Chemicon | MAB3152 | 1:100 |

TABLE 14

Secondary antibodies source and dilutions

| Reactive Sp. | Host | Fluorophore | Company | Catalog # | Dilution |
|---|---|---|---|---|---|
| Rabbit | Donkey | AlexaFluor 488 | Jackson | 715-546-150 | 1:100 |
| Goat | Donkey | AlexaFluor 647 | Jackson | 705-606-147 | 1:100 |
| Goat | Donkey | AlexaFluor 488 | Jackson | 705-546-147 | 1:100 |
| Rabbit | Donkey | AlexaFluor 594 | Jackson | 711-585-152 | 1:100 |
| Chicken | Donkey | AlexaFluor 488 | Jackson | 703-545-155 | 1:100 |

### Cardiac Differentiation of Human hPSC

Cells were seeded on Matrigel in mTeSR-1 medium and allowed to reach 80-90% confluence. Cardiac differentiation was conducted as previously described (Burridge, P. W. et al. Chemically defined generation of human cardiomyocytes. Nat Methods 11, 855-860, 2014). Briefly, a basal medium named CDM3 was used (RPMI-1640, 500 µg/mL recombinant human serum albumin, 213 µg/mL L-ascorbic acid 2-phosphate and 1% penicillin/streptomycin). At day zero of differentiation, the medium was replaced with CDM3 supplemented with 6 µmol/L CHIR99021 (Stemgent) for two days. At day two of the differentiation the culture-medium was switched to CDM3 medium supplemented with 2 µM Wnt-C59 (Selleckchem), for additional two days. From day four onward, cells were cultured with RPMI supplemented with B27-I (B27 supplement without Insulin; Gibco, USA). Then, at day 8-9, once the cells started beating, medium was replaced to CDM3 medium supplemented with 100 µM bovine serum albumin (control) or 100 µM oleic acid-albumin (Gibco, USA) and 50 µM 9CLA (Sigma, USA) and maintained for an additional 4 days. Media was supplemented with 10 µM GW9662 (Sigma Aldrich, USA) during inhibition studies as indicated in text. Additional details are provided in Tables 4-6 above.

### Quantification of Metabolic Function

Mitochondrial function was measured using the Seahorse XF Cell Mito Stress Test Kit according to the manufacturer’s instructions (Agilent, Santa Clara, CA). Briefly, cells were trypsinized, centrifuged for 5 minutes at 90 x g, re-suspended with control medium and seeded on Seahorse XFp miniplates coated with 1% Matrigel at a density of 3,000 cells per well. Cells were allowed to acclimate for 24 hours without fatty acids to remove transient and residual effects. Cultures were then incubated in unbuffered XF Base Medium supplemented with 2 mM Glutamine, 1 mM sodium pyruvate, and 10 mM glucose (pH 7.4) for 1 hour at 37° C. in a non-CO₂ incubator. The basal oxygen consumption rate (OCR) was measured for 30 minutes and then injected 1 µM oligomycin, a mitochondrial complex V inhibitor that blocks oxidative phosphorylation. The decrease in OCR due to oligomycin treatment was defined as the oxidative phosphorylation rate. 0.5 µM carbonyl cyanide 4-(trifluoromethoxy) phenylhydrazone (FCCP), an uncoupling agent, was added at 60 minutes to measure maximal mitochondrial activity, and complete inhibition was induced at 90 minutes using a mixture of 0.5 µM antimycin A and rotenone, mitochondrial complex III and mitochondrial complex I inhibitors.

### Example 1

### Microbiome-Derived Lipids Drive the Metabolic Maturation of the Heart

### Microbiome Derived Lipids Drive the Metabolic Maturation of the Heart

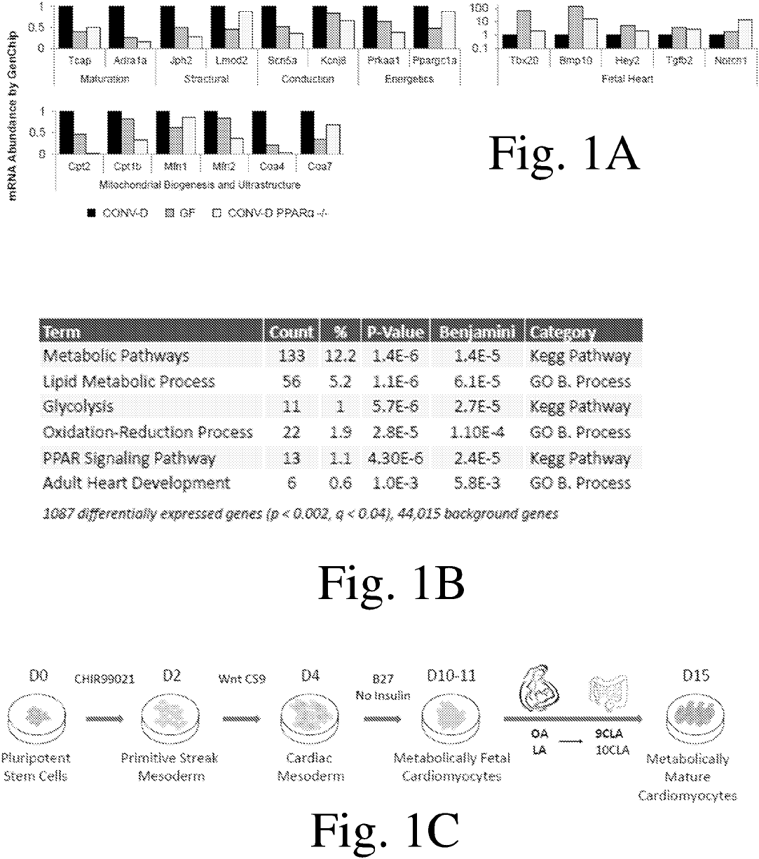

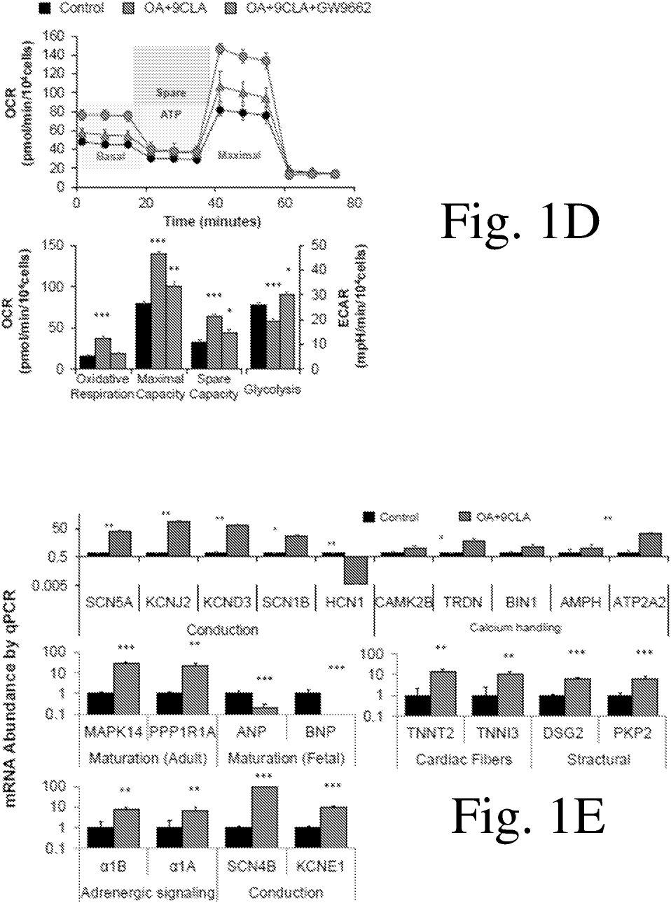

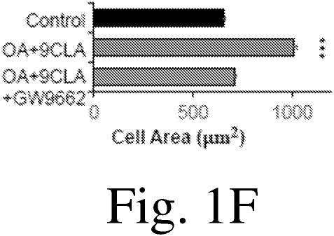

The heart matures rapidly postpartum, as the neonatal heart starts supporting the energetic demands of the growing infant. To enable the increased workload, oxidative capacity rises rapidly, as cardiomyocytes shift from glucose to fatty acids as a carbon source ⁵³. Analysis of mRNA isolated from cardiac tissue of 6- to 10-week old germ-free mice (GF) or conventionalized mice (CONV-D) that received a microbiota transplant, showed lower levels of mature structural, functional and metabolic genes, as well as elevated markers of fetal heart tissue suggesting retarded maturation in the germ free mice as compared to the conventionalized mice (FIG. 1A; FIGS. 2). Heart tissue of PPARα^{-/-} knockout animals showed a similar signature as the germ-free mice. Gene ontology analysis comparing GF to CONV-D mice showed disruption of metabolic pathways, including lipid metabolism and mitochondrial processes (FIG. 1B; FIGS. 2), pointing to the PPAR signaling pathway (q=2.4×10⁻⁵).

### Example 2

### In Vitro Human Cardiac Maturation

To model cardiac development in humans the present inventors utilized pluripotent stem cells derived cardiomyocytes that often show neonatal characteristics (FIG. 1C). Immunofluorescence analysis shows that treatment with 50 µM of OA and 9CLA mimicking postnatal nutrition for 4 days increased mitochondrial abundance and length, producing a mature interconnected network (data not shown). This effect was associated with a 3.5-folds increase in abundance of cardiac troponin I3 (TNNI3) fibers and sarcomeres, intertwined along the mitochondrial network (data not shown) Cardiomyocyte cell populations matured using the methods disclosed herein are homogenous and exhibit sarcomeres of 2.0 to 2.4 µm, typically about 2.1 µm. Fatty acid stimulation mimicking postnatal nutrition increased the oxidative phosphorylation of stem-cell derived cardiomyocytes, as well as maximal respiratory capacity by 1.8- to 2.5-fold (FIG. 1D). Glycolysis decreased by 24%, and cell area increased by 1.5-fold (hypertrophy), indicating of a more mature phenotype ⁵³. Furthermore, gene expression analysis showed up-regulation of key genes related to structural, functional and metabolic maturation of human cardiac tissue, as well as down-regulation of fetal markers (FIG. 1E). In all cases, the effect of postnatal lipids was reversed following GW9662 treatment supporting a PPAR-dependent mechanism. Computational analysis showed 9CLA-induced gene clusters strongly associated with in vivo heart maturation in mice (FIGS. 2).

### Analysis and Discussion

Mammalian development occurs primarily in utero but was shown to continue in the weeks and months after birth as the organism adapts to its changing environment ^{54,55}. While development is thought to be driven primarily by transcriptional network dynamics ¹⁻⁴, recent work demonstrated that environmental cues including gut colonization ⁵⁶ and nutrition ⁵⁷ could affect postnatal development. Previous studies demonstrated that microbiome-modified bile acids activate drug metabolism in fetal human hepatocytes (Avior, Y. et al. Microbial-derived lithocholic acid and vitamin K2 drive the metabolic maturation of pluripotent stem cells-derived and fetal hepatocytes. Hepatology 62, 265-278, 2015), while others showed that interactions between the microbiome and microglial cells supports mouse brain development ¹⁵ .

As human models are unavailable, germ-free (GF) animals can offer some insight into the interplay between the microbiome and neonatal organ development ^{58,59}. Previous studies show GF mice have an improperly developed abdominal cavity, decreased fertility, and less than 30% of the cardiac output of conventional animals ⁶⁰. Studies have thus far focused on microbiome development and its effects on adult animal and human metabolism ⁶¹⁻⁶⁴. The present study focuses on neonatal organ development, asking how the microbiome affects the metabolic and functional maturation of tissues after birth.

To gain insight into the interplay between the microbiome and postnatal organ development, the present inventors studied the heart of GF mice 4 to 6 weeks post-partum, once breast-feeding and maturation are complete ^{65,66}. Tissues isolated from GF mice showed fetal signatures, lack of mitochondrial development, and transcriptional signatures suggesting disruption of lipid metabolism, particularly linoleic acid and PPARα signaling (FIGS. 1A,B). Linoleic acid is abundant in human breast milk (Table 2) and is metabolized by Bifidobacterium and lactobacillus strains to 9CLA in the first days of life ⁴³. 9CLA was shown to be an agonist of PPARα, increasing insulin sensitivity and lipid peroxidation in obese adults ⁶⁷, while decreasing low density lipoproteins (LDL) in healthy individuals ⁶⁸. However, previous work showed that treating 3 week-old rats with 9CLA did not have a beneficial effect (Turpeinen, A. M. et al. Effects of cis-9,trans-11 CLA in rats at intake levels reported for breast-fed infants. Lipids 41, 669-677 (2006)), while administration of PPARα agonist fenofibrate actually retarded AFP silencing in neonatal rats ⁷⁰. These results mark the difficulty of using rodent models to study human development.

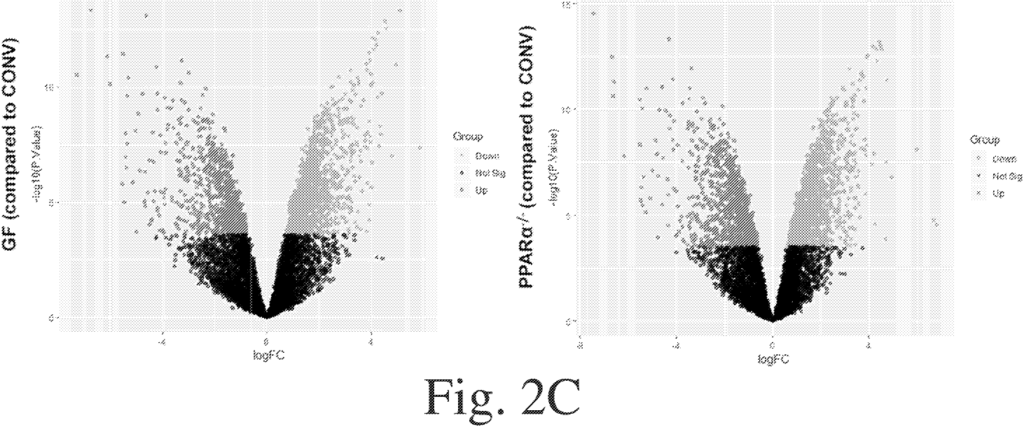

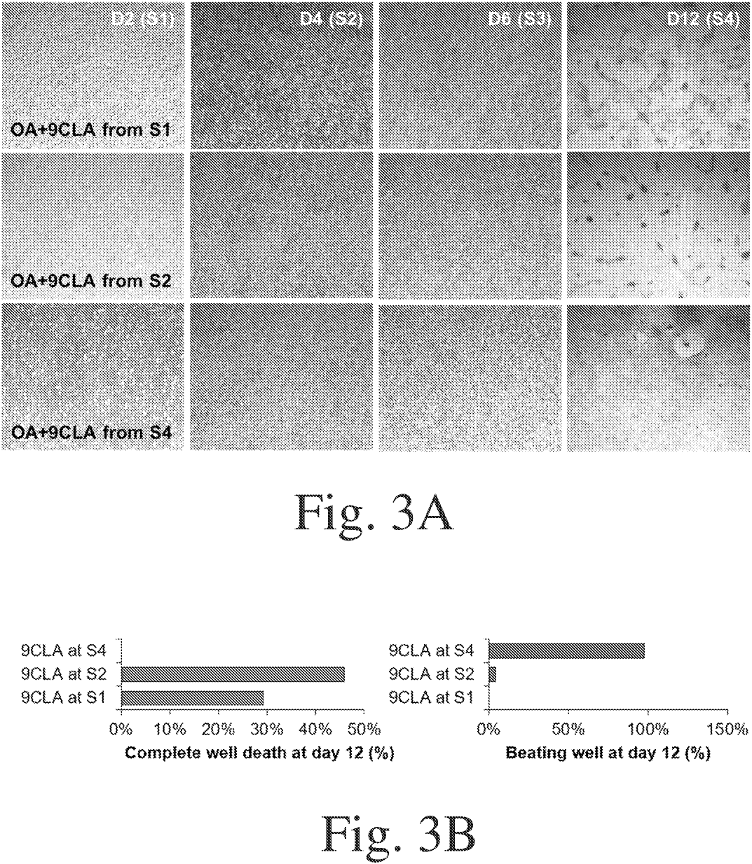

Human embryonic stem cells offer a fascinating model for the study of human development. The present inventors show that 9CLA addition drives dramatic mitochondrial development, transcriptional and functional maturation of cardiomyocytes via PPAR signaling (FIGS. 1, 2 and 3).

The careful orchestration of nutrition, physiology and organ development is intriguing. In utero, glycolysis can drive rapid embryo growth in a relatively low oxygen environment. Cardiac output and other organ function are minimal as the lungs and gut are mostly inactive. Post-partum increase in respiration is sudden, elevating oxygen supply and with it, cardiac output. Increase in organ perfusion flush some support cells, while requiring the organ to respond to the demands of the newly colonizing gut. Enteral nutrition is rich in milk glycans and lactate on which the bifidobacterium and lactobacillus thrive, driving these strains to dominance during postnatal lactation ³⁹⁻⁴². While both oleic and linoleic acids prime maturation, it is the absorption of microbiome derived 9CLA that pushes mitochondrial biogenesis and completes the shift toward oxidative phosphorylation. It is this metabolic shift during the first weeks of life that allows the neonate to continue its growth outside the uterus in a much more demanding but oxygen rich environment. This understanding could help customize neonatal nutrition, addressing risks associated with changes to the microbiome due to disease or antibiotic treatment common with preterm and high-risk labors ⁷².

It is important to note that current differentiation protocols of human pluripotent stem cells still produce glycolytic cells with many fetal characteristics ^{24,25}. This difficultly may be due to a missing segment in current protocols mimicking the postnatal state, not only in cardiac differentiation, but also in the derivation of oocytes, neurons, myocytes or pancreatic β-cells and other post-natal maturing cell types ⁷³⁻⁷⁶.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES (ADDITIONAL REFERENCES ARE CITED IN TEXT)

1 Wilson, M. E., Scheel, D. & German, M. S. Gene expression cascades in pancreatic development. Mechanisms of Development 120, 65-80, (2003).

2 Herriges, M. & Morrisey, E. E. Lung development: orchestrating the generation and regeneration of a complex organ. Development 141, 502-513, doi:10.1242/dev.098186 (2014).

3 Jiang, X. & Nardelli, J. Cellular and molecular introduction to brain development. Neurobiol Dis 92, 3-17, doi:10.1016/j.nbd.2015.07.007 (2016).

4 Gordillo, M., Evans, T. & Gouon-Evans, V. Orchestrating liver development. Development (Cambridge, England) 142, 2094-2108, doi:10.1242/dev.114215 (2015).

5 Ladle, D. R., Pecho-Vrieseling, E. & Arber, S. Assembly of Motor Circuits in the Spinal Cord: Driven to Function by Genetic and Experience-Dependent Mechanisms. Neuron 56, 270-283, (2007).

6 Ohler, U. & Wassarman, D. A. Promoting developmental transcription. Development 137, 15-26, doi:10.1242/dev.035493 (2010).

7 Hay, W. W., Jr. Placental-fetal glucose exchange and fetal glucose metabolism. Trans Am Clin Climatol Assoc 117, 321-340 (2006).

8 Hawdon, J. M. Postnatal metabolic adaptation and neonatal hypoglycaemia. Paediatrics and Child Health 26, 135-139, (2016).

9 Hommes, F. A., Kraan, G. P. B. & Berger, R. The Regulation of ATP Synthesis in Fetal Rat Liver. Enzyme and Protein 15, 351-360, doi:10.1159/000481072 (1973).

10 Pollak, J. K. & Sutton, R. The differentiation of animal mitochondria during development. Trends in Biochemical Sciences 5, 23-27, doi:10.1016/S0968-0004(80)80073-9 (1980).

11 Ogata, E. S. Carbohydrate Metabolism in the Fetus and Neonate and Altered Neonatal Glucoregulation. Pediatric Clinics of North America 33, 25-45, (1986).

12 Avior, Y. et al. Microbial-derived lithocholic acid and vitamin K2 drive the metabolic maturation of pluripotent stem cells-derived and fetal hepatocytes. Hepatology 62, 265-278, doi:10.1002/hep.27803 (2015).

13 Gury-BenAri, M. et al. The Spectrum and Regulatory Landscape of Intestinal Innate Lymphoid Cells Are Shaped by the Microbiome. Cell 166, 1231-1246.e1213, doi:10.1016/j.cell.2016.07.043 (2016).

14 Lahiri, S. et al. The gut microbiota influences skeletal muscle mass and function in mice. Science Translational Medicine 11, eaan5662, doi:10.1126/scitranslmed.aan5662 (2019).

15 Matcovitch-Natan, O. et al. Microglia development follows a stepwise program to regulate brain homeostasis. Science 353, aad8670, doi:10.1126/science.aad8670 (2016).

16 Martinez, M. Tissue levels of polyunsaturated fatty acids during early human development. The Journal of Pediatrics 120, S129-S138, (1992).

17 Suzuki, H., Park, S. J., Tamura, M. & Ando, S. Effect of the long-term feeding of dietary lipids on the learning ability, fatty acid composition of brain stem phospholipids and synaptic membrane fluidity in adult mice: a comparison of sardine oil diet with palm oil diet. Mech Ageing Dev 101, 119-128 (1998).

18 Park, E. J., Suh, M. & Clandinin, M. T. Dietary ganglioside and long-chain polyunsaturated fatty acids increase ganglioside GD3 content and alter the phospholipid profile in neonatal rat retina. Invest Ophthalmol Vis Sci 46, 2571-2575, doi:10.1167/iovs.04-1439 (2005).

19 Lauritzen, L. et al. DHA Effects in Brain Development and Function. Nutrients 8, 6, doi:10.3390/nu8010006 (2016).

20 Dvash, T., Ben-Yosef, D. & Eiges, R. Human embryonic stem cells as a powerful tool for studying human embryogenesis. Pediatr Res 60, 111-117, doi:10.1203/01.pdr.0000228349.24676.17 (2006).

21 Pera, M. F. & Trounson, A. O. Human embryonic stem cells: prospects for development. Development 131, 5515-5525, doi:10.1242/dev.01451 (2004).

22 Murry, C. E. & Keller, G. Differentiation of Embryonic Stem Cells to Clinically Relevant Populations: Lessons from Embryonic Development. Cell 132, 661-680, (2008).

23 Cohen, D. E. & Melton, D. Turning straw into gold: directing cell fate for regenerative medicine. Nature Reviews Genetics 12, 243, doi:10.1038/nrg2938 (2011).

24 Varum, S. et al. Energy Metabolism in Human Pluripotent Stem Cells and Their Differentiated Counterparts. PLOS ONE 6, e20914, doi:10.1371/journal.pone.0020914 (2011).

25 Machiraju, P. & Greenway, S. C. Current methods for the maturation of induced pluripotent stem cell-derived cardiomyocytes. World J Stem Cells 11, 33-43, doi:10.4252/wjsc.v11.i1.33 (2019).

26 Cox, L. M. et al. Altering the intestinal microbiota during a critical developmental window has lasting metabolic consequences. Cell 158, 705-721, doi:10.1016/j.cell.2014.05.052 (2014).

27 Nobel, Y. R. et al. Metabolic and metagenomic outcomes from early-life pulsed antibiotic treatment. Nat Commun 6, 7486, doi:10.1038/ncomms8486 (2015).

28 Fu, Z. D., Selwyn, F. P., Cui, J. Y. & Klaassen, C. D. RNA-Seq Profiling of Intestinal Expression of Xenobiotic Processing Genes in Germ-Free Mice. Drug Metab Dispos 45, 1225-1238, doi:10.1124/dmd.117.077313 (2017).

29 Selwyn, F. P., Cheng, S. L., Klaassen, C. D. & Cui, J. Y. Regulation of Hepatic Drug-Metabolizing Enzymes in Germ-Free Mice by Conventionalization and Probiotics. Drug Metab Dispos 44, 262-274, doi:10.1124/dmd.115.067504 (2016).

30 Weger, B. D. et al. The Mouse Microbiome Is Required for Sex-Specific Diurnal Rhythms of Gene Expression and Metabolism. Cell metabolism 29, 362-382.e368, doi:10.1016/j.cmet.2018.09.023 (2019).

31 Thaiss, C. A. et al. Microbiota Diurnal Rhythmicity Programs Host Transcriptome Oscillations. Cell 167, 1495-1510.e1412, doi:10.1016/j.cell.2016.11.003 (2016).

32 Cani, P. D. et al. Microbial regulation of organismal energy homeostasis. Nature Metabolism 1, 34-46, doi:10.1038/s42255-018-0017-4 (2019).

33 Moreno-Carranza, B. et al. Prolactin regulates liver growth during postnatal development in mice. Am J Physiol Regul Integr Comp Physiol 314, R902-R908, doi:10.1152/ajpregu.00003.2018 (2018).

34 Schmucker, D. L. Hepatocyte fine structure during maturation and senescence. Journal of Electron Microscopy Technique 14, 106-125, doi:10.1002/jemt.1060140205 (1990).

35 Wanet, A., Arnould, T., Najimi, M. & Renard, P. Connecting Mitochondria, Metabolism, and Stem Cell Fate. Stem Cells Dev 24, 1957-1971, doi:10.1089/scd.2015.0117 (2015).

36 Chen, X. et al. Differential reactivation of fetal/neonatal genes in mouse liver tumors induced in cirrhotic and non-cirrhotic conditions. Cancer Sci 106, 972-981, doi:10.1111/cas.12700 (2015).

37 Chen, C., Soto-Gutierrez, A., Baptista, P. M. & Spee, B. Biotechnology Challenges to In Vitro Maturation of Hepatic Stem Cells. Gastroenterology 154, 1258-1272, doi:10.1053/j.gastro.2018.01.066 (2018).

38 Levy, G. et al. Nuclear receptors control pro-viral and antiviral metabolic responses to hepatitis C virus infection. Nature Chemical Biology 12, 1037, (2016).

39 Conway, G. S. & Jacobs, H. S. Leptin: a hormone of reproduction. Hum Reprod 12, 633-635, doi:10.1093/humrep/12.4.633 (1997).

40 Haarman, M. & Knol, J. Quantitative real-time PCR assays to identify and quantify fecal Bifidobacterium species in infants receiving a prebiotic infant formula. Appl Environ Microbiol 71, 2318-2324, doi:10.1128/aem.71.5.2318-2324.2005 (2005).

41 Haarman, M. & Knol, J. Quantitative real-time PCR analysis of fecal Lactobacillus species in infants receiving a prebiotic infant formula. Appl Environ Microbiol 72, 2359-2365, doi:10.1128/aem.72.4.2359-2365.2006 (2006).

42 Sela, D. A. & Mills, D. A. The marriage of nutrigenomics with the microbiome: the case of infant-associated bifidobacteria and milk. Am J Clin Nutr 99, 697s-703s, doi:10.3945/ajcn.113.071795 (2014).

43 Devillard, E., McIntosh, F. M., Duncan, S. H. & Wallace, R. J. Metabolism of linoleic acid by human gut bacteria: different routes for biosynthesis of conjugated linoleic acid. J Bacteriol 189, 2566-2570, doi:10.1128/JB.01359-06 (2007).

44 Bugge, A. & Mandrup, S. Molecular Mechanisms and Genome-Wide Aspects of PPAR Subtype Specific Transactivation. PPAR Research 2010, doi:10.1155/2010/169506 (2010).

45 Pavek, P. Pregnane X Receptor (PXR)-Mediated Gene Repression and CrossTalk of PXR with Other Nuclear Receptors via Coactivator Interactions. Front Pharmacol 7, 456-456, doi:10.3389/fphar.2016.00456 (2016).

46 Leesnitzer, L. M. et al. Functional Consequences of Cysteine Modification in the Ligand Binding Sites of Peroxisome Proliferator Activated Receptors by GW9662. Biochemistry 41, 6640-6650, doi:10.1021/bi0159581 (2002).

47 Abass, K. et al. Characterization of human cytochrome P450 induction by pesticides. Toxicology 294, 17-26, (2012).

48 Li, H. & Wang, H. Activation of xenobiotic receptors: driving into the nucleus. Expert Opin Drug Metab Toxicol 6, 409-426, doi:10.1517/17425251003598886 (2010).

49 Chaffey, N. Alberts, B., Johnson, A., Lewis, J., Raff, M., Roberts, K. and Walter, P. Molecular biology of the cell. 4th edn. Ann Bot 91, 401-401, doi:10.1093/aob/mcg023 (2003).

50 Palakkan, A. A., Nanda, J. & Ross, J. A. Pluripotent stem cells to hepatocytes, the journey so far. Biomed Rep 6, 367-373, doi:10.3892/br.2017.867 (2017).

51 Fomin, M. E., Beyer, A. I. & Muench, M. O. Human fetal liver cultures support multiple cell lineages that can engraft immunodeficient mice. Open Biol 7, doi:10.1098/rsob.170108 (2017).

52 Guo, J. et al. Humanized mice reveal an essential role for human hepatocytes in the development of the liver immune system. Cell Death & Disease 9, 667, doi:10.1038/s41419-018-0720-9 (2018).

53 Piquereau, J. & Ventura-Clapier, R. Maturation of Cardiac Energy Metabolism During Perinatal Development. Front Physiol 9, 959, doi:10.3389/fphys.2018.00959 (2018).

54 Raznahan, A., Greenstein, D., Lee, N. R., Clasen, L. S. & Giedd, J. N. Prenatal growth in humans and postnatal brain maturation into late adolescence. Proc Natl Acad Sci USA 109, 11366-11371, doi:10.1073/pnas.1203350109 (2012).

55 Lopaschuk, G. D. & Jaswal, J. S. Energy metabolic phenotype of the cardiomyocyte during development, differentiation, and postnatal maturation. J Cardiovasc Pharmacol 56, 130-140, doi:10.1097/FJC.Ob013e3181e74a14 (2010).

56 Diaz Heijtz, R. et al. Normal gut microbiota modulates brain development and behavior. Proc Natl Acad Sci U S A 108, 3047-3052, doi:10.1073/pnas.1010529108 (2011).

57 Correia, C. et al. Distinct carbon sources affect structural and functional maturation of cardiomyocytes derived from human pluripotent stem cells. Scientific Reports 7, 8590, doi:10.1038/s41598-017-08713-4 (2017).

58 Al-Asmakh, M. & Zadjali, F. Use of Germ-Free Animal Models in Microbiota-Related Research. J Microbiol Biotechnol 25, 1583-1588, doi:10.4014/jmb.1501.01039 (2015).

59 Kennedy, E. A., King, K. Y. & Baldridge, M. T. Mouse Microbiota Models: Comparing Germ-Free Mice and Antibiotics Treatment as Tools for Modifying Gut Bacteria. Frontiers in Physiology 9, doi:10.3389/fphys.2018.01534 (2018).

60 Coates, M. E. & Gustafsson, B. E. The germ-free animal in biomedical research. (Laboratory Animals Ltd., 1984).

61 Kindt, A. et al. The gut microbiota promotes hepatic fatty acid desaturation and elongation in mice. Nature Communications 9, 3760, doi:10.1038/s41467-018-05767-4 (2018).

62 Morrison, D. J. & Preston, T. Formation of short chain fatty acids by the gut microbiota and their impact on human metabolism. Gut Microbes 7, 189-200, doi:10.1080/19490976.2015.1134082 (2016).

63 Semova, I. et al. Microbiota regulate intestinal absorption and metabolism of fatty acids in the zebrafish. Cell Host Microbe 12, 277-288, doi:10.1016/j.chom.2012.08.003 (2012).

64 Berger, C. N. et al. Citrobacter rodentium Subverts ATP Flux and Cholesterol Homeostasis in Intestinal Epithelial Cells In Vivo. Cell Metab 26, 738-752.e736, doi:10.1016/j.cmet.2017.09.003 (2017).

65 König, B. & Markl, H. Maternal care in house mice. Behavioral Ecology and Sociobiology 20, 1-9, doi:10.1007/BF00292161 (1987).

66 Dutta, S. & Sengupta, P. Men and mice: Relating their ages. Life Sciences 152, 244-248, (2016).

67 Riserus, U., Vessby, B., Ärnlöv, J. & Basu, S. Effects of cis-9,trans-11 conjugated linoleic acid supplementation on insulin sensitivity, lipid peroxidation, and proinflammatory markers in obese men. The American Journal of Clinical Nutrition 80, 279-283, doi:10.1093/ajcn/80.2.279 (2004).

68 Tricon, S. et al. Opposing effects of cis-9,trans-11 and trans-10,cis-12 conjugated linoleic acid on blood lipids in healthy humans. The American Journal of Clinical Nutrition 80, 614-620, doi:10.1093/ajcn/80.3.614 (2004).

69 Turpeinen, A. M. et al. Effects of cis-9,trans-11 CLA in rats at intake levels reported for breast-fed infants. Lipids 41, 669-677 (2006).

70 Staels, B. & Auwerx, J. Perturbation of developmental gene expression in rat liver by fibric acid derivatives: lipoprotein lipase and alpha-fetoprotein as models. Development 115, 1035-1043 (1992).

71 Miyajima, A. et al. Role of Oncostatin M in hematopoiesis and liver development. Cytokine Growth Factor Rev 11, 177-183 (2000).

72 Gasparrini, A. J. et al. Persistent metagenomic signatures of early-life hospitalization and antibiotic treatment in the infant gut microbiota and resistome. Nat Microbiol, doi:10.1038/s41564-019-0550-2 (2019).

73 Kaufman, B. A., Li, C. & Soleimanpour, S. A. Mitochondrial regulation of β-cell function: maintaining the momentum for insulin release. Mol Aspects Med 42, 91-104, doi:10.1016/j.mam.2015.01.004 (2015).

74 Kann, O. & Kovacs, R. Mitochondria and neuronal activity. American Journal of Physiology-Cell Physiology 292, C641-C657, doi:10.1152/ajpcell.00222.2006 (2007).

75 Son, G. & Han, J. Roles of mitochondria in neuronal development. BMB Rep 51, 549-556, doi:10.5483/BMBRep.2018.51.11.226 (2018).

76 Babayev, E. & Seli, E. Oocyte mitochondrial function and reproduction. Curr Opin Obstet Gynecol 27, 175-181, doi:10.1097/GCO.0000000000000164 (2015).

77 Michal Amit, M. K. C., Margaret S. Inokuma, Choy-Pik Chiu, Charles P. Harris, Michelle A. Waknitz, Joseph Itskovitz-Eldor, James A. Thomson. Clonally derived human embryonic stem cell lines maintain pluripotency and proliferative potential for prolonged periods of culture. Dev Biol 227, 271-278 (2000).

78 Chad A. Cowan, I. K., Jill McMahon, Jocelyn Atienza, Jeannine Witmyer, Jacob P. Zucker, Shunping Wang, Cynthia C. Morton, Andrew P. McMahon, Doug Powers, and Douglas A. Melton. Derivation of Embryonic Stem-Cell Lines from Human Blastocysts. N Engl J Med 350, 1353-1356 (2004).

79 James A. Thomson, J. I.-E., Sander S. Shapiro, Michelle A. Waknitz, Jennifer J. Swiergiel, Vivienne S. Marshall, Jeffrey M. Jones. Embryonic Stem Cell Lines Derived from Human Blastocysts. Science 282, 1145-1147 (1998).

80 Muschet, C. et al. Removing the bottlenecks of cell culture metabolomics: fast normalization procedure, correlation of metabolites to cell number, and impact of the cell harvesting method. Metabolomics 12, 151, doi:10.1007/s11306-016-1104-8 (2016).

81 Burridge, P. W. et al. Chemically defined generation of human cardiomyocytes. Nat Methods 11, 855-860, doi:10.1038/nmeth.2999 (2014).

82 Levy, G. et al. Long-term culture and expansion of primary human hepatocytes. Nat Biotechnol 33, 1264-1271, doi:10.1038/nbt.3377 (2015).

What is claimed is:

1. An in vitro method of generating a metabolically mature human cell in xeno-free media comprising:
(a) incubating a human stem or progenitor cell in differentiation medium for an effective amount of time to induce differentiation of said stem cell into an immature differentiated cell, in the absence of a trans fatty acid;
(b) incubating said immature differentiated cell of step (a) for a suitable time period, in a maturation medium comprising an effective amount of conjugated trans fatty acid to induce metabolic maturation, said treatment producing metabolically mature human cells containing trans fatty acids.

2. The method of claim 1, wherein said maturation medium further comprises a fatty acid selected from the group consisting of a monounsaturated omega-9 fatty acid, palmitic acid, linoleic acid (LA), or a short chain fatty acid, wherein said human stem or progenitor cell is an embryonic stem cell or an induced pluripotent stem cell.

3. The method of claim 1, wherein said trans fatty acid is cis-9, trans-11 conjugated linoleic acid (9CLA).

4. The method of claim 1, wherein said immature differentiated cell is a beating cardiomyocyte.

5. The method of claims 1, wherein incubation of said immature differentiated cell in said maturation medium increases spare mitochondrial capacity by at least 60%, as measured by seahorse assay.

6. The method of claim 1, wherein a metabolically mature cell is defined by spare mitochondrial capacity which is equal to, or greater than its basal respiration, as measured by seahorse assay and the cell of step a) is induced to differentiation by contact with a demethylation promoting agent.

7. The method of claim 1, wherein said human pluripotent stem cell is a human induced pluripotent stem cell derived from a somatic cell of an adult human subject being at least 8 years-old or is a human embryonic stem cell obtained following at least 50 passages.

8. The method of claim 2, wherein said monounsaturated omega-9 fatty acid is oleic acid (OA).

9. The method of claim 1, wherein said conjugated fatty acid is formed by the metabolism of human microbiome strains such as bifidobacterium and/or lactobacillus bacterial strain(s).

10. The method of claim 1, wherein said mitochondrial spare capacity is determined using a seahorse assay.

11. (canceled)

12. The method of claim 4, wherein said metabolically mature differentiated cell is a human cardiomyocyte exhibiting a mitochondrial network distributed in the cytosol of said cell which is not confined to the perinuclear space only as compared to said metabolically immature cardiomyocyte and said maturation medium comprises basal media supplemented with B27 supplement minus insulin (1X), oleic acid, and 9CLA.

13. The method of claim 12, wherein said metabolically mature differentiated human cardiomyocyte is characterized by
i) sarcomeres of 2.0 to 2.4 µm in length; and
ii) a reduced expression by at least 5 fold of a fetal marker selected from the group consisting of: Atrial natriuretic peptide (ANP), Brain Natriuretic Peptide (BNP), Hyperpolarization Activated Cyclic Nucleotide Gated Potassium Channel 1 (HCN1), MYH7 (myosin heavy chain 7), MYH6, cardiac titin (N2B), cardiac troponin I (TNNI3), and sarcoplasmic reticulum ATPase (SERCA2) as compared to the expression of said fetal marker in a human metabolically immature cardiomyocyte obtained in step (a) as measured by an QPCR or RNASEQ analysis.

14-15. (canceled)

16. The method of claim 1, wherein said effective concentration of said conjugated fatty acid in said culture medium is between 10-50 micromolar.

17. The method of claims 12 to 15, wherein said conjugated fatty acid is cis-9, trans-11 conjugated linoleic acid (9CLA).

18. The method of claims 12, wherein
i) said effective concentration of said monounsaturated omega-9 fatty acid is between 50-150 micromolar;
ii) said effective concentration of said oleic acid (OA) is between 50-150 micromolar;
iii) said effective concentration of said Palmitic Acid is between 50-150 micromolar;
iv) said effective concentration of said linoleic acid (LA) is between 50-150 micromolar;
v) said effective concentration of said short chain fatty acid is between 500-10,000 micromolar;
vi) said culture medium is devoid of serum;
vii) said culture medium is a chemically defined medium; and, or,
viii) said culture medium is devoid of supplemented Carnitine and wherein
at least two conditions from i) to viii) are present.

19-26. (canceled)

27. A metabolically mature human cardiomyocyte, having 9CLA present in cellular membranes, sarcomeres of 2.0 to 2.4 µm in length, and a mitochondrial spare capacity and a basal respiration rate of at least 60% above a spare capacity observed in fetal cardiomyocytes isolated from a human fetal heart obtained from a gestation week of 8 to 24 weeks, or an immature differentiated cell subjected to mitochondrial stress as measured by a seahorse assay, and a reduced expression by at least 5-fold of a fetal marker selected from the group consisting of: ANP, BNP, HCN1, and MIH6 as measured by an QPCR or RNASEQ analysis as compared to the expression of said fetal marker in said fetal cardiomyocyte or an isolated, homogeneous population of said cardiomyocytes.

28-32. (canceled)

33. A method of screening for a compound which modulates metabolic activity of a cell, comprising:
(a) incubating the isolated population of cells of claim 30 with the compound for a pre-determined time period, and;
(b) measuring following said pre-determined time period a level of metabolic activity selected from the group consisting of: an intracellular esterase activity and a conversion of MTT 3-(4,5-dimethylthiazol-2-yl)-2,5-diphenyltetrazolium bromide to insoluble formazan, wherein a decrease or an increase in said level below or above a predetermined threshold, respectively indicates that said compound is a modulator of the metabolic activity of the cell,
thereby screening for the compound which modulates metabolic activity of the cell.

34. The method of claim 33, wherein said incubation with said compound results in said decrease below said predetermined threshold or a decrease in a predetermined cell number, indicating said compound is toxic to cells.

35. The method of claim 34, wherein said incubation with said compound results in said increase above said predetermined threshold.

36-37. (canceled)

38. A kit for screening a compound which is toxic to cells, the kit comprising the isolated population of cells of claim 30 and at least one agent capable of detecting a toxicological end-point selected from the group consisting of: a cell viability assay, a functional viability assay, a calcium handling assay, an inflammation/injury marker assay or any other standard assay published by TOX21, EuroTOX, EPA or any other governmental agency.


SEQUENCE LISTING (44 sequences)

SEQ ID NO: 1 - DNA, 20 bp, Artificial sequence
  Single strand DNA oligonucleotide
tctgtttcccgtgctgcgag

SEQ ID NO: 2 - DNA, 19 bp, Artificial sequence
  Single strand DNA oligonucleotide
gcctcgtatgtgaggcaaa

SEQ ID NO: 3 - DNA, 20 bp, Artificial sequence
  Single strand DNA oligonucleotide
aaagtacgtccgcgggttgc

SEQ ID NO: 4 - DNA, 21 bp, Artificial sequence
  Single strand DNA oligonucleotide
aaggtgaagcgcaatgtccct

SEQ ID NO: 5 - DNA, 23 bp, Artificial sequence
  Single strand DNA oligonucleotide
tgctctgtgtcactgtggattgg

SEQ ID NO: 6 - DNA, 20 bp, Artificial sequence
  Single strand DNA oligonucleotide
tctccaggttgcctctcact

SEQ ID NO: 7 - DNA, 20 bp, Artificial sequence
  Single strand DNA oligonucleotide
acgagacagaagacggcatt

SEQ ID NO: 8 - DNA, 20 bp, Artificial sequence
  Single strand DNA oligonucleotide
gcttagcctcgtcgatgaac

SEQ ID NO: 9 - DNA, 21 bp, Artificial sequence
  Single strand DNA oligonucleotide
tctggagcagtattacgaccc

SEQ ID NO: 10 - DNA, 21 bp, Artificial sequence
  Single strand DNA oligonucleotide
tctcaggcatacgctgactac

SEQ ID NO: 11 - DNA, 21 bp, Artificial sequence
  Single strand DNA oligonucleotide
caacgcagacctgatggattt

SEQ ID NO: 12 - DNA, 20 bp, Artificial sequence
  Single strand DNA oligonucleotide
tggaaacgtccgggttacag

SEQ ID NO: 13 - DNA, 21 bp, Artificial sequence
  Single strand DNA oligonucleotide
acagagcggaaaagtgggaag

SEQ ID NO: 14 - DNA, 22 bp, Artificial sequence
  Single strand DNA oligonucleotide
cgtgtggacaaggtggatgaag

SEQ ID NO: 15 - DNA, 23 bp, Artificial sequence
  Single strand DNA oligonucleotide
gaagagttgagtgcagcacatac

SEQ ID NO: 16 - DNA, 22 bp, Artificial sequence
  Single strand DNA oligonucleotide
tttaatatttgcttgattgcag

SEQ ID NO: 17 - DNA, 20 bp, Artificial sequence
  Single strand DNA oligonucleotide
ggctccttctacctgcctct

SEQ ID NO: 18 - DNA, 20 bp, Artificial sequence
  Single strand DNA oligonucleotide
cctgaggatccattccaaga

SEQ ID NO: 19 - DNA, 21 bp, Artificial sequence
  Single strand DNA oligonucleotide
tcctgcgctatgagaatgagg

SEQ ID NO: 20 - DNA, 20 bp, Artificial sequence
  Single strand DNA oligonucleotide
tgtggcaggagacagttcag

SEQ ID NO: 21 - DNA, 20 bp, Artificial sequence
  Single strand DNA oligonucleotide
ctctttccacgatggctttg

SEQ ID NO: 22 - DNA, 20 bp, Artificial sequence
  Single strand DNA oligonucleotide
cgggtgtggcacagctagtt

SEQ ID NO: 23 - DNA, 20 bp, Artificial sequence
  Single strand DNA oligonucleotide
cccagcgtccagtagcacac

SEQ ID NO: 24 - DNA, 20 bp, Artificial sequence
  Single strand DNA oligonucleotide
cccattcgtagcctttggta

SEQ ID NO: 25 - DNA, 19 bp, Artificial sequence
  Single strand DNA oligonucleotide
tccgatgagtccggccagt

SEQ ID NO: 26 - DNA, 21 bp, Artificial sequence
  Single strand DNA oligonucleotide
cccccagctgctcaaaaatgc

SEQ ID NO: 27 - DNA, 21 bp, Artificial sequence
  Single strand DNA oligonucleotide
gggcaaagaggctggtcttca

SEQ ID NO: 28 - DNA, 20 bp, Artificial sequence
  Single strand DNA oligonucleotide
gtggaggaagctgacaacaa

SEQ ID NO: 29 - DNA, 19 bp, Artificial sequence
  Single strand DNA oligonucleotide
ccctctggatccactgctt

SEQ ID NO: 30 - DNA, 20 bp, Artificial sequence
  Single strand DNA oligonucleotide
aaccccaagatgcacaactc

SEQ ID NO: 31 - DNA, 21 bp, Artificial sequence
  Single strand DNA oligonucleotide
ctggctggaatctagcagtct

SEQ ID NO: 32 - DNA, 21 bp, Artificial sequence
  Single strand DNA oligonucleotide
ggagactctgtactcgaaggt

SEQ ID NO: 33 - DNA, 19 bp, Artificial sequence
  Single strand DNA oligonucleotide
agcccccgcttcttcattc

SEQ ID NO: 34 - DNA, 20 bp, Artificial sequence
  Single strand DNA oligonucleotide
ctgatccggtccatcttcct

SEQ ID NO: 35 - DNA, 21 bp, Artificial sequence
  Single strand DNA oligonucleotide
tcgttgatcctgtttcggaga

SEQ ID NO: 36 - DNA, 22 bp, Artificial sequence
  Single strand DNA oligonucleotide
gccgcttaaacttgcctcgaag

SEQ ID NO: 37 - DNA, 25 bp, Artificial sequence
  Single strand DNA oligonucleotide
cttgcttctactgtcaaagtctacg

SEQ ID NO: 38 - DNA, 22 bp, Artificial sequence
  Single strand DNA oligonucleotide
tgtcataggttttaggaacagg

SEQ ID NO: 39 - DNA, 20 bp, Artificial sequence
  Single strand DNA oligonucleotide
agggcttgaaatcagggaag

SEQ ID NO: 40 - DNA, 20 bp, Artificial sequence
  Single strand DNA oligonucleotide
ggttgaggcagctgttgaag

SEQ ID NO: 41 - DNA, 22 bp, Artificial sequence
  Single strand DNA oligonucleotide
tggtgttgtgctcgtagttttc

SEQ ID NO: 42 - DNA, 20 bp, Artificial sequence
  Single strand DNA oligonucleotide
gcttcttggagcggatgtag

SEQ ID NO: 43 - DNA, 20 bp, Artificial sequence
  Single strand DNA oligonucleotide
gtcaaggagctggaagtgct

SEQ ID NO: 44 - DNA, 22 bp, Artificial sequence
  Single strand DNA oligonucleotide
tgcattgtcaagtgacgatcac